(19) United States
(12) Patent Application Publication
(10) Pub. No.: US 2025/0305960 A1
(43) Pub. Date: Oct. 2, 2025

(54) SYSTEM AND METHOD FOR SPECTROSCOPIC DETERMINATION OF A CHEMOMETRIC MODEL FROM SAMPLE SCANS

(71) Applicant: THERMO SCIENTIFIC PORTABLE ANALYTICAL INSTRUMENTS INC., Tewksbury, MA (US)

(72) Inventors: Nimesh Khadka, Dracut, MA (US); Lin Zhang, Andover, MA (US); Jeneffer England, Tewksbury, MA (US)

(21) Appl. No.: 19/077,995

(22) Filed: Mar. 12, 2025

Related U.S. Application Data
(60) Provisional application No. 63/569,969, filed on Mar. 26, 2024.

(51) Int. Cl. G01N 21/65; G01N 33/92

(52) U.S. Cl. CPC G01N 21/65; G01N 33/92; G01N 2201/1296

(57) ABSTRACT

A computer-implemented method is provided. The method includes obtaining a chemometric model for one or more levels of parameters associated with composition training data. The composition training data includes data representative of one or more components of a vesicle. Raman spectra data representative of at least one Raman spectrum associated with one or more components of a sample of a vesicle is received. The Raman spectra data representative of the at least one Raman spectrum associated with the one or more components of the sample of the vesicle is transferred into the chemometric model. A level of one or more parameters of the one or more components of the sample of the vesicle is determined based on the chemometric model.

### RELATED APPLICATIONS

This application claims priority to U.S. Provisional Application No. 63/569,969, filed Mar. 26, 2024, the entire content of which is incorporated herein by reference.

### FIELD

The present disclosure generally relates to systems and methods for conducting spectroscopic analytical techniques, such as Raman spectroscopy. In particular, systems and methods are disclosed for determining a chemometric model for one or more levels of one or more parameters based on one or more components of a sample of a vesicle.

### BACKGROUND

Raman spectroscopy has scientific, commercial, and public safety applications. The stability and functionality for developing lipid nanoparticles (LNPs) are dependent on the quantity, types of lipids, and the ratio of individual lipids. The presence of impurities or degraded lipid components severely affects the quality of the LNPs. The current state-of-the-art uses methods and systems of mass spectrometry and high-performance liquid chromatography (HPLC) as analytical tools for analysis of LNPs. Use of mass spectrometry and HPLC require significant resources, such as a trained user, highly technical instrumentation and data analysis, and extensive time for analyzing samples of the LNPs.

### SUMMARY

According to one aspect of the present disclosure, a computer-implemented method on an analytical instrument support apparatus is disclosed. The method includes receiving, by one or more processors, Raman spectra data associated with composition training data (e.g., actual data), wherein the composition training data includes data representative of one or more components of a vesicle. One or more pre-processing operations are applied by one or more processors to standardize the Raman spectra data. A covariance matrix is determined by one or more processors based on, at least, the standardized Raman spectra data. One or more principal components values associated with the covariance matrix are determined by one or more processors. A chemometric model based on the one or more principal component values is determined by one or more processors for one or more levels of parameters associated with the data representative of the one or more components of the vesicle.

According to another aspect of the present disclosure, another computer-implemented method on an analytical instrument support apparatus is disclosed. The method includes obtaining, by one or more processors, a chemometric model for one or more levels of parameters associated with composition training data, wherein the composition training data includes data representative of one or more components of a vesicle. Raman spectra data representative of at least one Raman spectrum associated with one or more components of a sample of a vesicle is received by one or more processors. The Raman spectra data representative of the at least one Raman spectrum associated with the one or more components of the sample of the vesicle is transferred into the chemometric model by one or more processors. A level of one or more parameters of the one or more components of the sample of the vesicle based on the chemometric model is determined by one or more processors.

According to another aspect of the present disclosure, an analytical instrument support system is disclosed. The analytical instrument support system includes one or more computer processors, one or more non-transitory computer-readable storage media, and program instructions stored on at least the one or more non-transitory computer readable storage media for execution by at least one of the one of the one or more processors. The program instructions comprise: (i) program instructions to receive Raman spectra data associated with composition training data, wherein the composition training data includes data representative of one or more components of a vesicle; (ii) program instructions to apply one or more pre-processing operations to standardize the Raman spectra data; (iii) program instructions to determine a covariance matrix based on, at least, the standardized Raman spectra data; (iv) program instructions to determine one or more principal component values associated with the covariance matrix; and (v) program instructions to determine a chemometric model based on the one or more principal component values for one or more levels of parameters associated with the data representative of the one or more components of the vesicle.

According to another aspect of the present disclosure, an analytical instrument support system is disclosed. The analytical instrument support system includes one or more computer processors, one or more non-transitory computer-readable storage media, and program instructions stored on at least the one or more non-transitory computer readable storage media for execution by at least one of the one of the one or more processors. The program instructions comprise: (i) program instructions to obtain a chemometric model for one or more levels of parameters associated with composition training data, wherein the composition training data includes data representative of one or more components of a vesicle; (ii) program instructions to receive Raman spectra data representative of at least one Raman spectrum associated with one or more components of a sample of a vesicle; (iii) program instructions to transfer the Raman spectra data representative of the at least one Raman spectrum associated with the one or more components of the sample of the vesicle into the chemometric model; and (iv) program instructions to determine a level of one or more parameters of the one or more components of the sample of the vesicle based on the chemometric model.

According to another aspect of the present disclosure, an analytical instrument is disclosed. The analytical instrument includes a light source configured to direct onto a surface of a sample and a spectrograph configured to acquire a Raman spectrum from the surface of the sample in response to the light source directing light onto the surface of the sample, one or more processors, one or more non-transitory computer-readable storage media, and program instructions stored on at least one of the one or more non-transitory computer-readable storage media for execution by at least one of the one or more processors. Execution of the program instructions by at least one of the one or more processors cause the analytical instrument to implement the following acts, comprising: (i) program instructions to receive Raman spectra data associated with composition training data, wherein the composition training data includes data representative of one or more components of a vesicle; (ii) program instructions to apply one or more pre-processing operations to standardize the Raman spectra data; (iii) program instructions to determine a covariance matrix based on, at least, the standardized Raman spectra data; (iv) program instructions to determine one or more principal component values associated with the covariance matrix; and (v) program instructions to determine a chemometric model based on the one or more principal component values for one or more levels of parameters associated with the data representative of the one or more components of the vesicle.

According to another aspect of the present disclosure, an analytical instrument is disclosed. The analytical instrument includes a light source configured to direct onto a surface of a sample and a spectrograph configured to acquire a Raman spectrum from the surface of the sample in response to the light source directing light onto the surface of the sample, one or more processors, one or more non-transitory computer-readable storage media, and program instructions stored on at least one of the one or more non-transitory computer-readable storage media for execution by at least one of the one or more processors. Execution of the program instructions by at least one of the one or more processors cause the analytical instrument to implement the following acts, comprising: (i) program instructions to obtain a chemometric model for one or more levels of parameters associated with composition training data, wherein the composition training data includes data representative of one or more components of a vesicle; (ii) program instructions to receive Raman spectra data representative of at least one Raman spectrum associated with one or more components of a sample of a vesicle; (iii) program instructions to transfer the Raman spectra data representative of the at least one Raman spectrum associated with the one or more components of the sample of the vesicle into the chemometric model; and (iv) program instructions to determine a level of one or more parameters of the one or more components of the sample of the vesicle based on the chemometric model.

There is no specific requirement that a system, method, or technique relating to determination-based spectroscopy include all of the details characterized herein, in order to obtain some benefit according to the present disclosure. Thus, the specific examples characterized herein are meant to be exemplary applications of the techniques described, and alternatives are possible.

### BRIEF DESCRIPTION OF THE DRAWINGS

Features and advantages of the present technology will become more apparent from the following detailed description of exemplary embodiments thereof taken in conjunction with the accompanying drawings in which:

FIG. 1 is a block diagram of an exemplary analysis system, according to some implementations of the present disclosure.

FIG. 2 illustrates an optical architecture for a spectrometer included in the analysis system of FIG. 1, according to some implementations of the present disclosure.

FIG. 3 illustrates another optical architecture for the spectrometer included in the analysis system of FIG. 1, according to some implementations of the present disclosure.

FIG. 4 illustrates a further optical architecture for the spectrometer included in the analysis system of FIG. 1, according to some implementations of the present disclosure.

FIG. 5 illustrates yet another optical architecture for the spectrometer included in the analysis system of FIG. 1, according to some implementations of the present disclosure.

FIG. 6 is a flowchart of an exemplary process to determine a chemometric model based on principal component values for one or more levels of parameters associated with the data representative of components of the vesicle, according to some implementations of the present disclosure.

FIG. 7 is a flowchart of an exemplary process to determine a level of one or more parameters of one or more components of a sample of a vesicle based on an exemplary chemometric model, according to some implementations of the present disclosure.

FIG. 8 is a graphical illustration of exemplary Raman spectra data representative of at least one Raman spectrum associated with one or more components of an exemplary vesicle, according to some implementations of the present disclosure.

FIG. 9 illustrates performance of an exemplary chemometric model using principal component analysis (PCA) for determining one or more parameters representative of a quality of one or more components of a sample of a vesicle, according to some implementation of the present disclosure.

FIG. 10 is a graphical illustration of exemplary Raman spectra data representative of mono-component lipid quantification in an ethanol solution, according to some implementations of the present disclosure.

FIG. 11 illustrates performance of an exemplary chemometric model for determining one or more parameters representative of a quantity of isopropyl myristate in ethanol, according to some implementations of the present disclosure.

FIG. 12 illustrates the concentration of isopropyl myristate versus the concentration of cholesterol as performed according to some implementations of the present disclosure.

FIG. 13 is a graphical illustration of Raman spectra data representative of at least one Raman spectrum associated with one or more components of a vesicle, according to some implementations of the present disclosure.

FIG. 14 illustrates performance of an exemplary chemometric model for determining one or more parameters representative of a quantity of isopropyl myristate and cholesterol in a mixture with ethanol, according to some implementations of the present disclosure.

FIG. 15 illustrates performance of an exemplary chemometric model for determining one or more parameters representative of a quantity of isopropyl myristate and cholesterol in a mixture with ethanol, according to some implementations of the present disclosure.

FIG. 16 is a graphical illustration of the Raman spectra data of FIG. 13 illustrating data excluded from quantification models, according to some implementations of the present disclosure.

FIG. 17 is a graphical illustration of the Raman spectra data illustrating aqueous contamination quantification using a partial least squares (PLS) model, according to some implementations of the present disclosure.

FIG. 18 illustrates performance of an exemplary chemometric model for determining one or more parameters representative of a quantity of water, according to some implementations of the present disclosure.

While the present technology is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Unless otherwise defined, all technical and scientific terms used herein have the same meaning as commonly understood by one of ordinary skill in the art. In case of conflict, the present document, including definitions, will control. Example methods and systems are described below, although methods and systems similar or equivalent to those described herein can be used in practice or testing of the present disclosure. All publications, patent applications, patents and other references mentioned herein are incorporated by reference in their entirety. The systems, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Raman measurement” refers to a Raman system where the illumination spot diameter remains fixed-size and has a uniform radial distribution.

“Aspheric diffuse ring producing optic” refers to various implementations for producing the distributed spot which includes an aspheric diffuse ring producing optic, or ADRPO. In some implementations, aspheric optics may include what is referred to as an axicon or conical optic which produces a ring of intensity but has higher order aspheric terms to produce the spread-out pattern. In some implementations, the aspheric optic may have coefficients of A1=0.01, A2=0.06, and A4=0.002, with all other terms being zero.

“Collimating lens” refers to optical elements that transform the incoming light direction to parallel paths.

“Filter” refers to optical elements that remove some wavelengths of incoming light.

“Focusing optics” refers to optical elements that transform the incoming light direction to a point in space.

“Light source” refers to a light source used for excitation in spectroscopy application. Exemplary systems and methods may include a laser that is adapted for Raman spectroscopy such as 785 m, or 1064 nm. Exemplary light sources could also include a broad band source such as an LED. In some implementations, the laser power affects the values of the base value and the bright-max intensity values when a sample is scanned. Exemplary systems and methods disclose and contemplate using a wide range of laser power.

“Sample surface plane” refers to the surface of the sample under test where the illumination area is directed.

“Steering mirrors” refers to optical elements used to change the direction of light path.

“Raman spectra data” refers to a spectrum of data values that may be representative of a bright spectrum and/or a dark spectrum. Where the bright spectrum is the scattered light from the sample hitting a detector. The dark spectrum is a spectrum received when no light hits the detector. The dark spectrum captures the shape of the baseline offset.

Systems, methods, and techniques are disclosed herein for chemometric modeling that is applied to determine a level of one or more parameters of a sample, such as samples of physical compounds or biological substances. The present disclosure can be particularly desirable by providing chemometric model determinations that are rapidly obtainable relative to current state-of-the-art methods and systems. For example, the present disclosure provides improved methods for determining a level of one or more parameters of a sample based on at least one Raman spectrum of the sample. In some implementations, the present disclosure desirably provides improved methods and system for determining the level of the one or more parameters of the sample by utilizing continuous Raman spectroscopy scans during the lipid formulation, thereby improving the accuracy, and reducing the analysis time used during current state-of-the-art methods and systems. The improvements of the present disclosure include rapid and accurate continuous monitoring of concentration of individual lipids during lipid formulation, degraded lipid impurities, physical properties in solution, and aqueous contamination.

Improvements of the present disclosure may also be used to continuously monitor the formation of lipid nanoparticles (LNPs) and the formulated LNPs which are stored for future use. Current state-of-the-art methods and systems require increased time and cost to analyze and/or monitor the stored LNPs. The improvements of the present disclosure provide a rapid, accurate and continuous quality control measure by monitoring the LNPs to determine a level of one or more parameters of the stored LNPs prior to use.

As described herein, the Raman measurement parameters for the analytical system are initial targets provided as instructions to the analytical instrument for obtaining a spectrum. The Raman measurement parameters can include scan time and one or more Raman shift wavenumbers. The system and methods described herein model these Raman measurement parameters to determine a level of one or more parameters of one or more components of a sample of a vesicle based on a chemometric model. For example, a chemometric model is obtained for one or more levels of one or more parameters associated with composition training data, wherein the composition training data includes data representative of one or more components of a vesicle. The chemometric model determines a level of the one or more parameters of the sample based on, at least, transferring the Raman spectra data of the at least one Raman spectrum associated with the one or more components of the sample of the vesicle into the chemometric model.

Raman spectroscopy is an effective tool for identifying and characterizing various sample compounds and substances. In Raman spectroscopy, light typically from a laser and of a known wavelength (typically infrared or near infrared) is directed at a sample compound or substance. The laser light (also sometimes referred to as a Raman pump) interacts with the electron clouds in the molecules of the sample compound or substance and, as a result of this interaction, experiences selected wavelength shifting. The precise nature of this wavelength shifting depends upon the materials present in the sample compound or substance. A unique wavelength signature (typically called the Raman signature) is produced by each sample compound or substance. This unique Raman signature permits the sample compound or substance to be identified and characterized. More specifically, the spectrum of light returning from the sample compound or substance is analyzed with a spectrometer so as to identify the Raman-induced wavelength shifting in response to the Raman pump light, and then this wavelength signature is compared (e.g., by a computing device) with a library of known Raman signatures, whereby to identify the precise nature of the sample compound or substance.

The present disclosure is now described with reference to the drawings, wherein like reference numerals are used to refer to like elements throughout. In the following description, for purposes of explanation, numbers of specific details are set forth in order to provide an improved understanding of the present disclosure. It may be evident, however, that the systems and methods of the present disclosure may be practiced without one or more of these specific details. In other aspects, well-known structures and devices are shown in block diagram form in order to facilitate describing the systems and methods of the present disclosure.

It should be understood that although implementations are described herein as being used with a spectrometer or other optical instrument, implementations can be constructed as stand-alone devices for measuring an electrochemical property of a sample compound or substance. Furthermore, although some implementations are described herein with respect to measuring an electrochemical property of a sample compound or substance, exemplary methods and systems described herein can be used to measure other electrochemical properties, such as, for example a Raman spectrum of the sample compound or substance.

Exemplary analysis systems can be used in a variety of environments to identify unknown materials, to monitor the production and/or formulation of compositions within an enclosed or open environment, to evaluate the threat posed by unknown materials, to provide positive identification of packaged raw materials, or to provide general security screening functions of a variety of substances. Exemplary analysis systems can include a wide range of sizes, from portable, handheld instruments to larger systems in permanent laboratories.

### Exemplary Analysis Systems

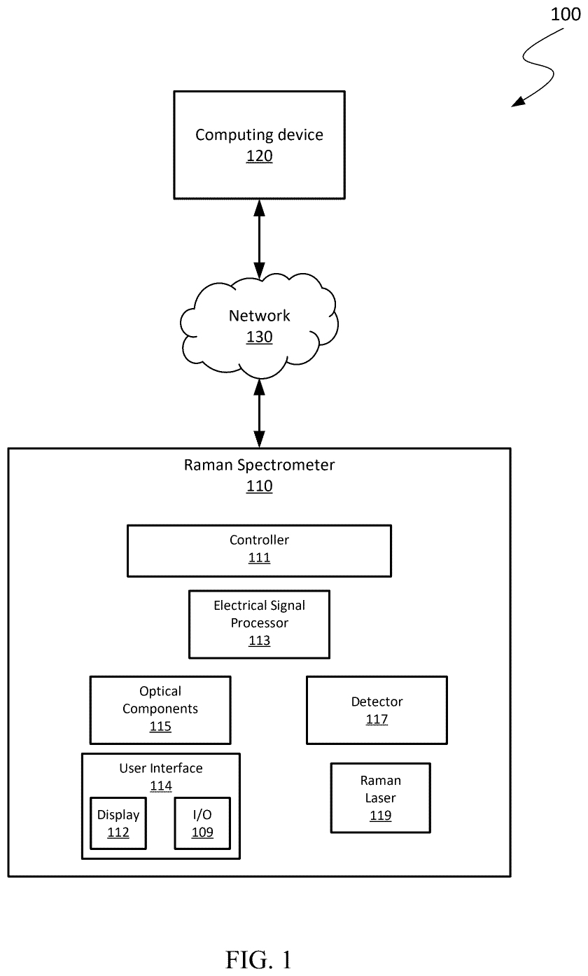

Those of ordinary skill in the art appreciate that there are a variety of different optical architectures and arrangements utilized in the field of Raman spectroscopy. FIG. 1 provides an illustrative example of an analysis system 100 (also referred to herein as “analyzer 100”) that comprises an optical architecture and other elements that operate to measure one or more Raman spectra from a sample via one or more of the methods described herein.

The analyzer 100 illustrated in FIG. 1 includes a spectroscopic system 110 communicatively coupled to a computing device 120 via a network 130. As illustrated in FIG. 1, the spectroscopic system 110 includes a controller 111, an electronic signal processor 113, and a spectrometer 140 (e.g., a Raman spectrometer).

It will be appreciated that, in some implementations, at least a portion of the computing device 120 may be located separate from the spectroscopic system 110, which provides the opportunity for increased computing power at a central location or across multiple locations. One skilled in the art can envision various interconnections, both physical and wireless, between the components of the analysis system 100. It will further be appreciated that, in some implementations, the spectroscopic system 110 and the computing device 120 may be communicatively coupled without the network 130 (e.g., via a dedicated wired or wireless connection). Alternatively, some implementations of the analyzer 100 may not require the resources of computing device 120 but may instead utilize resources internal to the spectroscopic system 110 to perform the methods described herein. Thus, computing device 120 may not be necessary for operation of the analyzer 100 and/or the spectroscopic system 110 and the example of FIG.1 should not be considered as limiting. As described herein, the analyzer 100 may be used to measure one or more Raman spectra from a sample compound or substances via one or more of the methods described herein.

It should be understood that, in some implementations, the components of the analyzer 100 and/or the spectroscopic system 110 illustrated in FIG. 1 may be included in a common housing forming an analytical instrument that may include a benchtop or a portable Raman spectrometer device (e.g., a handheld device). However, in other implementations, one or more components of the analyzer 100 and/or the spectroscopic system 110 may be contained in separate housings or devices and may be coupled (e.g., communicatively, electrically, mechanically, or the like) as needed to carry out the methods described herein. Also, in some implementations, the operations described herein as being performed by the components of the analyzer 100 and/or the spectroscopic system 110 may be combined and distributed in various ways. For example, in some implementations, an electronic signal processor 113 may be part of a controller 111, wherein the controller 111 is configured to perform the operations of the electrical signal processor 113 as described herein. Furthermore, the operations described herein as being performed by the controller 111 may be distributed among multiple controllers. In the same or alternative examples, operations described herein as being performed by controller 111 may be distributed among one or more computing devices (e.g., the processor 113, the computing device 120, or multiple computing devices). In some implementations, the controller 111 is configured to control operation of the spectrometer 140, wherein the processor 113 is configured to control other components of the spectroscopic system 110 (e.g., communication with the computing device 120). However, these roles of the controller 111 and the processor 113 may be combined and distributed in various ways, and, in some implementations, the spectroscopic system 110 includes only the controller 111 or the processor 113 and the included devices performed the functionality of both the controller 111 and the processor 113 as described herein.

The spectroscopic system 110 may also include additional components (such as power components), a user interface 114 (such as a display 112 and/or user input and/or output (“I/O”) device 109, such as, for example, a keyboard, a mouse, a touch screen), optical components (e.g., mirrors, lens, fiber optic cables, gratings, and filters), and the like. The spectrometer 140 included in the spectroscopic system 110 includes one or more optical components 145, a detector 147 (e.g., a CCD detector, a PMT detector, or other detector known in the art), and a light source 149. The light source 149 provides an excitation beam (e.g., excitation Laser providing 785 nm or 1064 nm light) to a sample (not shown in FIG. 1).

As described above, the spectroscopic system 110 and/or the spectrometer 140 may comprises a fully integrated portable system operated by a user on battery power to take Raman spectroscopy measurements in a variety of environments, such as, for example, a laboratory setting, a manufacturing (e.g., bioreactor based) setting, a remote setting, etc. Also, in the same or alternative implementations, elements of the spectroscopic system 110 may be utilized as separated systems communicatively connected (e.g., optically, wirelessly, electrically, mechanical, and the like) operated on battery power and/or power outlets connected to a central power source to take Raman spectroscopy measurements in the variety of environments described.

Referring now to light source 149 of spectrometer 140, it will be appreciated that implementations of light source 149 may emit wavelengths of light as needed for an application, for example, including or between a range of about 400 nm to about 1064 nm, a range of about 400 nm to about 750 nm, a range of about 400 nm to about 600 nm, a range of about 400 nm to about 500 nm, a range of about 600 nm to about 900 nm, a range of about 700 nm to about 850 nm, a range of 600 nm to 1064 nm, a range of 750 nm to 1064 nm, a range of 850 nm to 1064 nm, a range of 950 nm to 1064 nm, as well as a wavelength of about 785 nm, or a wavelength of about 1064 nm.

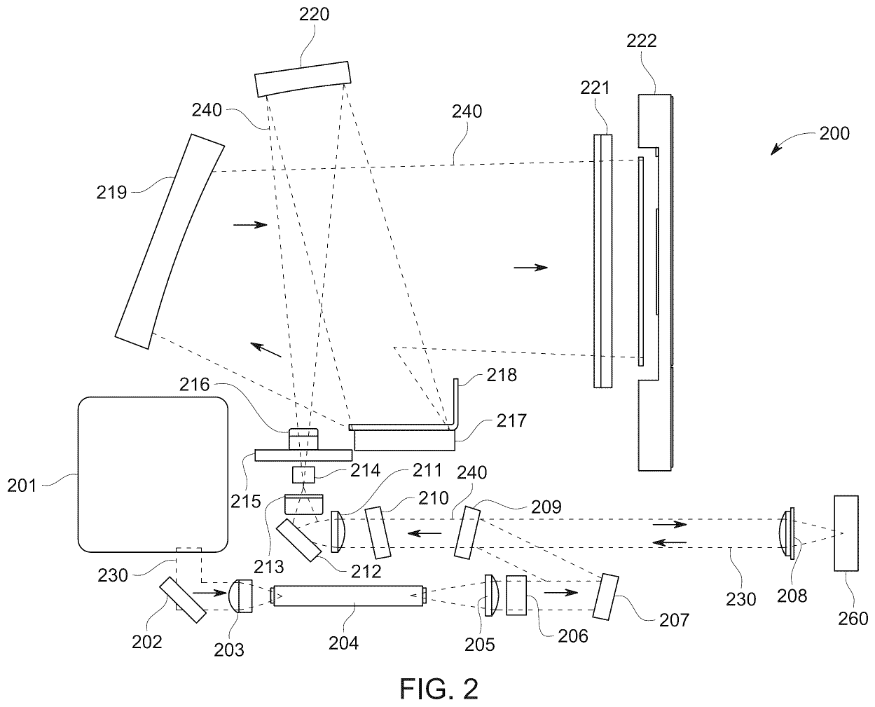

FIG. 2 provides an illustrative example of one implementation of an optical architecture comprising optical components of the spectrometer 140 (see FIG. 1), that are otherwise collectively referred to herein as an optical system 200. It will be appreciated that different optical architectures of Raman spectrometer are known in the art and thus the example of FIG. 2 should not be considered as limiting. For example, some implementations employ what are referred to as transmission gratings rather the reflection gratings, as well as associated differences in optical architecture.

The example of FIG. 2 illustrates one implementation of light source 149 (see FIG. 1) as laser assembly 201 comprising a laser source that produces a beam of light that travels along optical or beam path 230 (e.g., arrows illustrate direction of travel of the light beam) to sample 260. It will be appreciated that sample 260 may include any type of sample of interest to a user and may include substantially dry samples (e.g., a powder, solid material), substantially fluid samples (e.g., a liquid, gas), or some combination thereof (e.g., a gel). In response to the light from laser assembly 201, the sample 260 produces scattered light (e.g., comprising a Raman portion and a Rayleigh portion of scattered light), which travels along optional or beam path 240.

In some implementations, the laser assembly 201 may produce laser power as needed for an application for example, including or between a range of about 250 mW to about 750 mW; about 250 mW to about 700 mW; about 250 mW to about 650 mW; about 250 mW to about 600 mW; about 250 mW to about 550 mW; about 250 mW to about 500 mW; about 250 mW to about 450 mW; about 250 mW to about 400 mW; about 250 mW to about 350 mW; about 250 mW to about 300 mW; or about 250 mW. Also in some implementations, the laser power affects the values of the base value and the bright-max intensity values when sample 260 is scanned. It will be appreciated that other ranges and/or levels of laser power are known in the art and thus the example described for laser assembly 201 should not be considered as limiting.

FIG. 2 also illustrates one implementation of an architecture that directionally controls the beam path 230 and the beam path 240 as well as conditions one or more characteristics of the beam of light produced from the laser assembly 201 as well as from the sample 260. For example, a turning mirror 202 redirects beam path 230 to focusing lens 203 that focuses the beam onto a waveguide phase scrambler 204 (e.g., to adjust the phase characteristics of the beam). The beam exits waveguide phase scrambler 204 and travels to a collimating lens 205 (e.g., which adjusts collimation characteristics of the beam), then to a broadband filter 206 transmissive to a specific wavelength or range of wavelengths of light. The beam travels to a flat mirror 207 that redirects the beam path 230 to a selective clement 209. It will be appreciated that the selective element 209 may include a dichroic mirror, a notch filter, or other element that comprises substantially reflective characteristics to the wavelength(s) of the beam from laser assembly 201 and comprises substantially transmissive characteristics to a wavelength or wavelength range associated with Raman scattered light from sample 260. In the described example, selective element 209 redirects the beam path 230 to a lens 208 that focuses the beam to the sample 260. In the described example, the lens 208 may include any type of lens known in the art such as an objective lens that focuses the beam onto the sample 260. Also, some implementations of the lens 208 comprise special configurations and characteristics that provide advantages for different types of the sample 260 as will be described below.

The lens 208 collects Raman scattered light and Rayleigh scattered light produced from the sample 260 in response to the beam from the laser assembly 201 and produces the beam path 240 that travels back to the selective element 209 and a second selective element 210. As described above, the selective elements 209 and 210 are substantially transmissive to the wavelengths of the Raman scattered light, allowing the beam path 240 to pass through to additional optical elements that further adjust the path and conditions the characteristics of the beam traveling along the beam path 240. For example, the optical elements may include a focusing lens 211, a flat mirror 212, a baffle 213, a slit 214, a baffle 215, and a collimating lens 216.

The beam path 240 travels from the collimating lens 216 to a mirror 220 that reflects the beam path 240 toward a diffraction grating 217. It will be appreciated that, in the example of FIG. 2, the diffraction grating 217 comprises a reflective diffraction grating that produces a spectral distribution of light. The beam path 240 then travels to a focusing mirror 219 that redirects the beam path 240 to a focusing lens 221 that directs the beam to elements of a detector 222 (one implementation of the detector 147 of FIG. 1). It will also be appreciated that FIG. 2 illustrates a baffle 218 that, in some implementations, controls stray light.

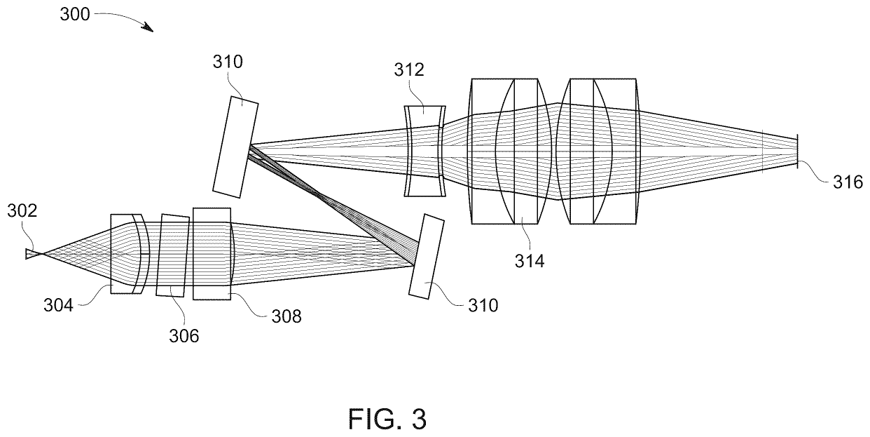

As described above, it will be appreciated that a variety of implementations of lens 208 are available that provide different focusing and light collection characteristics. For example, FIG. 3 provides an example implementation of an optical architecture useful for analyzing a sample contained in a package (e.g., a bag, bottle, etc.), where the optical architecture comprises some components of the optical system 200 (see FIG. 2) and other components that provide the characteristics of lens 208 (see FIG. 2), collectively referred to as an optical arrangement 300. In the described example, the optical arrangement 300 includes an element 302 that may include a focusing lens 203 (see FIG. 2) or an output from an optical fiber. Element 302 directs a beam (e.g., produced from light source 149 or laser assembly 201 or a Raman laser 119, 519—see FIGS. 1, 2, and 5) to a collimating lens 304 that produces a substantially collimated beam. In the described example, the collimating lens 304 can be movably mounted such that it can change position along the axis of the optical path. The range of motion includes a range of about 0.1 mm to about 10 mm to allow for a change in spot size on the sample surface to range from about 10 microns to about 10 mm. It will also be appreciated that in some implementations any of the collimating lens 304, a concave focusing lens 312, and/or focusing optics 314, either alone or in combination, may be movably mounted to effect a change in spot size.

The collimating lens 304 directs the substantially collimated beam into an aspheric diffuse ring producing optic 308 configured to produce a light pattern that is radially diffuse. The intensity of the output from the aspheric diffuse ring producing optic 308 is more intense at the outer edge of the resulting pattern than in the center. While this pattern could be projected directly onto a sample surface 316, in practical application it is advantageous to use one or more steering mirrors 310, one or more filters 306, and focusing elements, such as, for example, a concave focusing lens 312 and focusing optics 314, to direct the radially diffuse light pattern onto the sample surface 316.

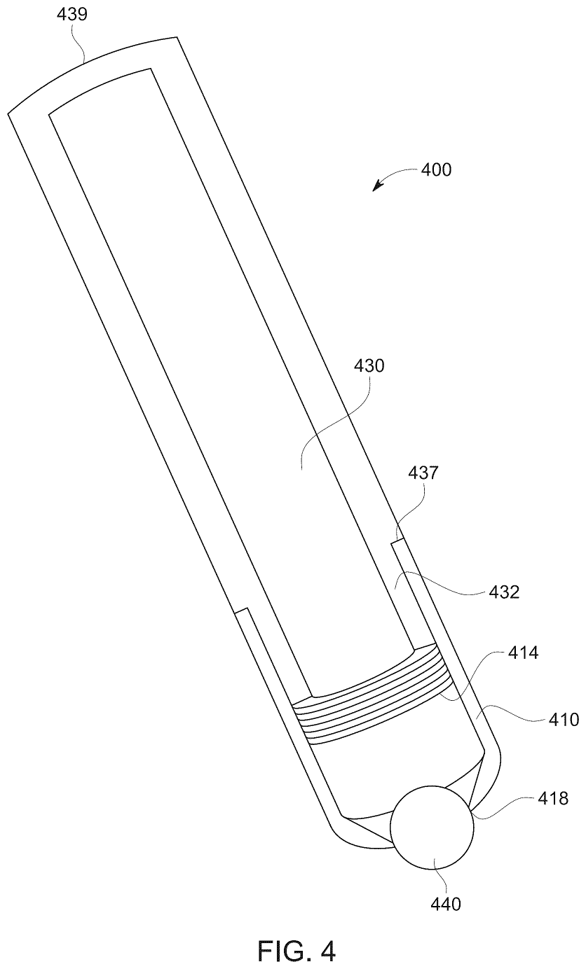

FIG. 4 provides an example of another implementation of the lens 208 (see FIG. 2), wherein this example may be useful for analyzing a fluid or semi-fluid sample. The implementation illustrated in FIG. 4 comprises some components of the optical system 200 and other components that provide characteristics of what is generally referred to as an “immersion probe,” wherein the components are collectively referred herein to as an optical arrangement 400. The implementation illustrated in FIG. 4 comprises a spherical lens 440 seated within a cylindrical probe tip 410 at lens opening 418. A seal between the probe tip 410 and the lens 440 is formed at the opening by any means known in the art, including all forms of welding or braising and the use of epoxies or other adhesives. The probe tip 410 may be any length. Optionally, the probe tip 410 may have threads 414 on its interior surface and may be extended using probe tube 430, which has threaded collar 432 for threading into probe tip 410. A seal is optionally formed between probe tube lip 437 and the distal end of probe tip 410. Further, in the described example, the optical arrangement 400 includes fiber optic coupling 439 that transmits illumination light from the laser assembly 201 (see FIG. 2) as well as scattered light from the sample 260 (see FIG. 2), wherein the sample 260 may include a liquid sample where lens 440 is immersed in the liquid. Also in the described example, the optical arrangement 400 may be configured as a separated element from spectroscopic system 110 (see FIG. 1) where an optical fiber provides optical communication between spectroscopic system 110 and the optical arrangement 400.

It will be appreciated that the examples provided in FIG. 3 and FIG. 4 are for the purposes of illustration and some implementations may include additional or fewer elements as needed for an application. For instance, in some implementations one or more windows, collimating lenses or other optical elements may be employed in applications that utilize a fiber optic coupling or other need for conditioning a beam or protecting internal environments. Therefore, the examples provided in FIG. 3 and FIG. 4 should not be considered as limiting.

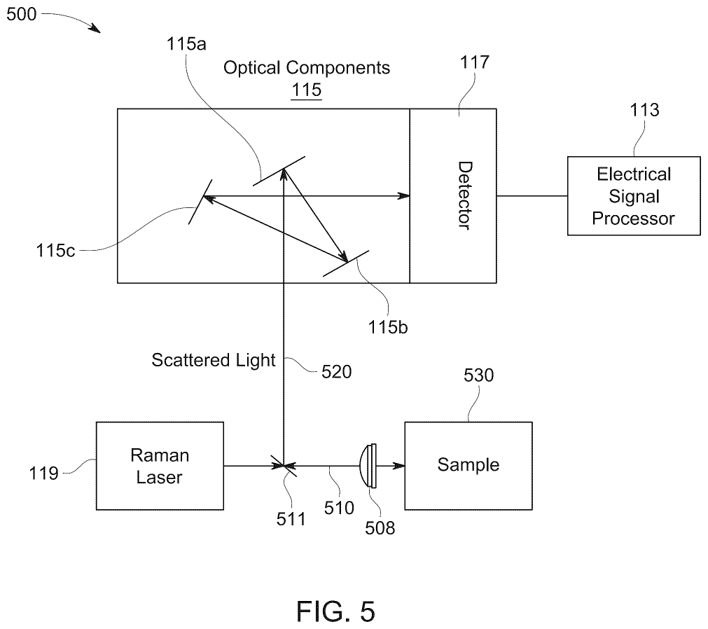

FIG. 5 provides another example of an implementation of an optical architecture comprising optical components of the spectrometer 140 (see FIG. 1), that are otherwise collectively referred to herein as the optical system 500. It will be appreciated that different optical architectures of Raman spectrometer are known in the art and thus the example of FIG. 5, similar to the examples of FIGS. 1 to 4, should not be considered as limiting.

The example of FIG. 5 illustrates one implementation of the light source 149 (see FIG. 1) as a Raman laser 119 comprising a laser source that produces a beam of light that travels along a first optical or beam path 510 (e.g., arrows illustrate direction of travel of the light beam) to a sample 530. Like sample 260 (see FIG. 2), it will be appreciated that sample 530 may include any type of sample of interest to a user which may include substantially dry samples (e.g., a powder, solid material), substantially fluid samples (e.g., a liquid, gas), or some combination thereof (e.g., a gel). In response to the light from the Raman laser 119, the sample 530 produces scattered light along a second optical or beam path 520 (e.g., comprising a Raman portion and a Rayleigh portion of scattered light).

In some implementations, the Raman laser 119 may produce laser power as needed for an application for example, including or between a range of about 250 mW to about 1050 mW, including various subranges therebetween such as the non-limiting subranges described above for the light source 149 and the laser assembly 201. It will also be appreciated that in some implementations, the laser power affects the values of the base value and the bright-max intensity values when the sample 530 is scanned.

FIG. 5 illustrates an architecture that in some implementations directionally controls the first beam path 510 and/or the second beam path 520. In some implementations, the beam paths 510, 520 can be controlled using one or more of turning mirrors, waveguide phase scramblers, various lenses, broadband filters, or selective elements (e.g., mirrors, notch filters, or other elements with substantially reflective characteristics to the wavelength(s) of the beam from the Raman laser 119 and/or substantially transmissive characteristics to a wavelength or wavelength range associated with Raman scattered light from sample 530). In the described example, a selective clement 511 is transmissive to the laser wavelengths emitted from the Raman laser 119 allowing the first beam path 510 to be directed to a lens 508 that focuses the beam onto the sample 530. In the described example, the lens 508 may include any type of lens known in the art such as an objective lens or lens architecture such as used in the optional arrangements 300 or 400 (see FIG. 3 and FIG. 4) that focuses the beam onto the sample 530.

Some implementations of the lens 508 include special configurations and characteristics that provides advantages for different types of samples. For example, the lens 508 can collect Raman scattered light and Rayleigh scattered light produced from the sample 530 in response to the beam from the Raman laser 119. The scattered light collected by the lens 508 is directed back from the surface of the sample 530 and travels back along the first beam path 510 to the selective element 511 (e.g., a beam splitter, such as, for example, a dichroic mirror) that directs the scattered light along the second beam path 520. In some implementations, the selective element 511 is substantially reflective to the wavelengths of the Raman scattered light, allowing the second beam path 520 to be directed to additional optical elements that further adjust the path and condition the characteristics of the beam traveling along the second beam path 520. Other optical arrangements are also contemplated for the selective element 511 for directing the scattered light along the second beam path 520.

As illustrated in FIG. 5, the optical system 500 also includes one or more optical components 115 (also referred herein as optical components 115a-115c), which can include one or more of collimating lens and mirrors, filters, such as, for example, a notch filter, diffraction gratings, and/or mirror relays. The scattered light is directed by one or more of optical components 115a-115c onto a detector 117 (an implementation of the detector 147 of FIG. 1). Signal processing and/or digitizing of signals associated with the scattered light that is received by the detector 117 is performed by an electrical signal processor associated with optical system 500, which may be, for example, the processor 113, the controller 111, the computing device 120, or a combination thereof. For example, in some implementations, the electrical signal processor 113 may be a suitably programmed microprocessor or application specific integrated circuit including a read-only or read-write memory of any known type which holds instructions and data for spectrometer operation as described herein.

As described above, it will be appreciated that a variety of implementations of the lens 508 are available that provide different focusing and light collection characteristics.

Returning to FIG. 1, the computing device 120 may be a standalone device, a server, internet of things (IoT), a laptop computer, a tablet computer, a netbook computer, a personal computer (PC), a smartphone, a personal digital assistant (PDA), a desktop computer, or any programmable electronic device capable of receiving, sending, and processing data. In some implementations, the computing device 120 includes one or more processors, one or more input/output processors, and one or more memory or data storage devices. In some implementations, the computing device 120 also includes one or more input/output devices, such as, for example, a display, a touchscreen, a keyboard, a mouse, or the like, which may be used to provide calibration or setting options to a user for operating the spectroscopic system, to provide analysis results to a user, or a combination thereof.

Similarly, the controller 111 (see FIG. 1) may include an electronic processor, an input/output (I/O) interface, and a data storage device (not shown); however, it should be understood that the controller 111 may have additional or fewer components. The controller 111 is suitable for the application and setting, and can include, for example, multiple electronic processors, multiple I/O interfaces, multiple data storage devices, or combinations thereof. In some implementations, some or all of the components included in the controller 111 may be attached to one or more mother boards and enclosed in a housing (e.g., including plastic, metal and/or other materials). In some implementations, some of these components may be fabricated onto a single system-on-a-chip, or SoC (e.g., an SoC may include one or more processing devices and one or more storage devices).

As used herein, “processors” or “electronic processor” or “electronic signal processor” refers to any device(s) or portion(s) of a device that process electronic data from registers and/or memory to transform that electronic data that may be stored in registers and/or memory. The electronic processor included in the controller 111 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The data storage device(s) included in the controller 111 may include one or more local or remote memory devices such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some implementations, the data storage device(s) may include memory that shares a die with a processor. In such an embodiment, the memory may be used as a cache memory and may include embedded dynamic random-access memory (eDRAM) or spin transfer torque magnetic random-access memory (STT-MRAM), for example. In some implementations, the data storage device may include non-transitory computer readable media having instructions thereon that, when executed by one or more processors (e.g., the electronic processor included in the controller 111), causes the controller 111 to store various applications and data for performing one or more of the methods described herein or portions described herein. For example, one or more data storage devices may store a modeling program, parameter and component data, chemometric model data, composition training data, or a combination thereof. Alternatively, or in addition, this program and the associated data or a portion thereof may be stored and executed on the computing device 120. Also, it should be understood that each method described herein may be implemented via one application or multiple applications.

In some implementations, the modeling program may be configured to execute one or more sets of program instructions to perform one or more operations and/or processes, such as, for example, access, retrieve, receive, identify, analyze, determine, and/or generate the parameter component data, the chemometric model data, and/or the composition training data.

In some implementations, the parameter component data may include data of one or more exemplary components associated with a vesicle. In some implementations, the vesicle may be a lipid nanoparticle (LNP). The lipid nanoparticles (LNPs) may also include one or more exemplary components. The one or more exemplary components may include one or more solvents, one or more lipids, one or more surfactants, one or more LNPs, one or more micelles, one or more stabilizers (e.g., hydrating agents such as glycerin or propylene glycol), one or more buffers, one or more salts, one or more waxes and/or one or more intermediate phases of an LNP stored on the data storage device.

In some implementations, the parameter and component data may include data of one or more parameters representative of a quality of the one or more components of the vesicle stored on the data storage device. In some implementations, the one or more parameters representative of the quality of the one or more exemplary components may indicate an identity of the one or more exemplary components modeled by a chemometric model, as described herein. In some implementations, the identity associated with the one or more exemplary components may include one or more solvents, one or more lipids, one or more surfactants, one or more LNPs, one or more micelles, one or more stabilizers (e.g., hydrating agents such as glycerin or propylene glycol), one or more buffers, one or more salts, one or more waxes and/or one or more intermediate phases of an LNP, or combinations thereof, as will be described in further detail below.

In some implementations, the parameter component data may include data of one or more parameters representative of a quantity of the one or more exemplary components of the vesicle stored on data storage device. In some implementations, the one or more parameters representative of the quantity of the one or more exemplary components of a vesicle may indicate an amount of the one or more exemplary components modeled by the chemometric model, as described herein. In some implementations, the amount of the one or more exemplary components may include a concentration such as mass concentration, molar concentration, volume concentration, and number concentration. In some implementations. the amount of the one or more exemplary components may include a ratio of the one or more components of the vesicle.

In some implementations, the quantity associated with the one or more parameters may indicate that the one or more components of the vesicle have degraded and/or that the vesicle has been contaminated (e.g., aqueous contamination). In some implementations, the quantity associated with the one or more parameters may indicate a concentration of each individual lipid, a concentration of each individual solvent, a concentration of each individual surfactant, a concentration of each individual LNP, a concentration of each individual micelle, or a concentration of each individual intermediate phase of an LNP. In some implementations, the quantity associated with the one or more parameters may indicate one or more degraded lipid impurities. In some implementations, the quantity associated with the one or more parameters may include physical properties (e.g., monomers and aggregation) of any one of solvents, lipids, surfactants, LNPs, micelles, intermediate phases of an LNP, or any combination thereof. In some implementations, the quantity associated with the one or more parameters may indicate an aqueous contamination of the vesicle.

In some implementations, the chemometric model data may include any number of regression analyses or machine learning processes including partial least squares regression (PLS) model, partial least squares discriminant analysis (PLSDA), principal component analysis (PCA) model, principal component regression (PCR) model, least absolute shrinkage and selection operator (LASSO) model, elastic-net regression model, support vector machine (SVM) model, neural network model, or combinations thereof stored on the data storage device. A brief description of the use of these regression analyses or machine learning processes are described below.

In some implementations, the above-described regression analyses and/or machine learning processes may be implemented on one or more processors. The one or more processors may be included in the controller 111 and/or a third-party computing device (e.g., the computing device 120) communicatively connected to spectroscopic system 110.

In some implementations, the PCA is an unsupervised statistical model, also known as singular value decomposition. It may learn to model a training data set on the testing data set, performing outlier detection on these principal components to find which samples belong to the same distribution as the training data set. This may be, therefore, a one-class classification model. The principal components can be computed by doing an eigen decomposition of the covariance matrix of the data. The eigenvectors with the highest corresponding eigenvalues then represent most of the variance in the data. This creates an orthogonal space in which the data can be represented. The main hyperparameter here is the number of eigenvectors k that are used to represent the data. Using more eigenvectors will give a higher explained variance of the model. Two statistical tests may then be used to identify outliers, the Hotelling T² test and Q residuals test. The Hotelling T² test focuses on the distance of the sample in principal component space to the rest of the sample, while the Q-test focuses on the residuals between the sample and a reconstruction of the sample after being transformed to PC-space and back. These tests are complementary to each other, and if either of the tests classifies the sample as an outlier, in some implementations, the systems disclosed herein may consider the sample an outlier. Because PCA is a dimensionally reduction algorithm, it can also be used as a pre-processing step for other models. The reduced dimensionality may lead to less overfitting on the training data.

In some implementations, the PLS or Partial Least Squares regression (also known as “Projection to Latent Structures”) is a statistical method that generalizes and combines features from principal component analysis and multiple regression. It can be useful to predict a set of dependent variables from a very large set of independent variables (i.e., predictors). The goal of PLS regression is to predict Y and X and to describe their common structure. When Y is a vector and X is full rank, this goal may be accomplished using ordinary multiple regression. When the number of predictors is large compared to the number of observations, X is likely to be singular and the regression approach is no longer feasible (i.e., because of multicollinearity).

In some implementations, the PLSDA is an adaption of PLS for categorical target variables. The procedure here is similar to PCA, in the sense that a dimensionality reduction is performed to obtain scores and loadings, but for PLS the decompositions are done in such a way that the covariance between predictors and targets is maximized in these scores. On the scores, a regression algorithm can be trained to predict the predictors. In PLSDA, the target variables are given as one-hot encoded vectors, for which the regression can be calculated.

In some implementations, the SVM is utilized for binary classification, where a selection is made between two classes. SVM is linear and attempts to construct a hyperplane in feature space that maximally separates the training datapoints based on their class. Classification then involves checking on which side of the hyperplane a new testing point is and assigning the corresponding class. By using kernels, the SVM can become increase operating strength. These kernels allow for non-linear transformations, meaning that non-linear decision surfaces can be constructed. Each kernel has its own set of hyperparameters that allow for further tuning of the model. Whereas the basic SVM is for binary classification, it can be extended to also allow for multi-class classification. In some implementations, the SVM may be preceded by a PCA decomposition to prevent or limit over fitting, as described above. An SVM can also be utilized as a one-class model for outlier detection. In this case, the SVM is trained on a data set that only contains samples of the class that are to be identified. A minimal envelope is then constructed as hyperplane around this data set in feature space. Any new test point outside of the envelope is classified as an outlier. This model can be used as a stand-alone one-class model for authentication, or as an outlier model, in addition to a multi-class classifier. In some implementations, for the one-class SVM, no dimensionality reduction may be used. Such one-class SVMs may perform well on high-dimensional data in the systems disclosed herein without the use of PCA for feature extraction.

In some implementations, the LASSO or Least Absolute Shrinkage and Selection Operator is a statistical formula for the regularization of data models and feature selection. It is used over regression methods for a more accurate prediction. The model uses shrinkage, where data values are shrunk towards a central point as the mean. The LASSO procedure provides for simple, sparse models (i.e., models with fewer parameters). This particular type of regression is well suited for models showing high levels of multicollinearity or for automating certain types of model selection, such as variable selection/parameter elimination.

In some implementations, the Elastic Net method overcomes the limitations of the LASSO method which uses a penalty function based on equation 1, show below:

$${‖ β ‖}_{1} = {\sum }_{j = 1}^{p} \vert {β}_{j} \vert \qquad ( 1 )$$

Use of equation 1, as shown above, has several limitations. In some implementations, when high-dimensional data with few examples are provided, the LASSO selects at most n variables before it saturates. Also, if there is a group of highly correlated variables, then then LASSO tends to select one variable from a group and ignores the other variables. To overcome these limitations, the clastic net adds a quadratic part to the penalty, which when used alone is ridge regression (also known as Tikhonov regularization).

In some implementations, the composition training data may include any number of Raman spectra, parameters, components, vesicles, one or more experimental designs, or combinations thereof stored on the data storage device. In some implementations, the plurality of composition training data is used to train any one of the one or more chemometric models, as described herein. In some implementations, the composition training data is stored on the composition training data.

In some implementations, the one or more experimental designs may include unform design, factorial design, fractional factorial design, response surface design, random design, block design, Box-Wilson Central Composite Design, Box-Behnken Design, or combinations thereof. In some implementations, the experimental designs may include uniform design.

In some implementations, the one or more experimental designs are provided as a process for the composition training data for carrying out research in an objective and controlled environment. Where precision is maximized, and specific conclusions may be drawn regarding one or more hypothesis statements. The one or more experimental designs may establish the effect that one or more factors or independent variables have on one or more dependent variables.

In some implementations. the I/O interface of the controller 111 may include one or more communication chips, connectors, and/or other hardware and software to govern communications between the controller 111 and other components. The I/O interface may include interface circuitry for coupling to the one or more components using any suitable interface (e.g., a Universal Serial Bus (USB) interface, a High-Definition Multimedia Interface (HDMI) interface, a Controller Area Network (CAN) interface, a serial Peripheral Interface (SPI) interface, an Ethernet interface, a wireless interface, or any other appropriate interface). For example, I/O interface may include circuitry for managing wireless communications for the transfer of data to and from the controller 111. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although, in some implementations the associated devices might not. Circuitry included in the I/O interface for managing wireless communications may implement any of a number of wireless standards or protocols, including but not limited to Institute for Electrical Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some implementations, circuitry included in the I/O interface for managing wireless communications may operate in accordance with a Global System for Mobile Communication (GSM), General Packet Radio Service (GPRS), Universal Mobile Telecommunications System (UMTS), High Speed Packet Access (HSPA), Evolved HPS (E-HPSA), or LTE network. In some implementations, circuitry included in the I/O interface for managing wireless communications may operate in accordance with Enhanced data for GSM Evolution (EDGE), GSM EDGE Radio Access Network (GERAN), Universal Terrestrial Radio Access Network (UTRAN), or Evolved UTRAN (E-UTRAN). In some implementations, circuitry included in the I/O interface for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some implementations, the I/O interface may include one or more antennas (e.g., one or more antenna arrays) for receipt and/or transmission of wire communications.

In some implementations, the analysis system 100 provides a stand-alone or dedicated analytical instrument or device (or set of instruments or devices) configured to perform a short scan and analysis of a sample. However, in other implementations, the analysis system 100 may be configured to perform additional scans or analysis of one or more samples. Combining such scanning abilities or analysis in one system (e.g., one analytical instrument) creates desirable efficiencies and improved accuracy in the analysis as multiple scans can be taken from the sample without having to change the position of the sample, reconfigure the analytical instrument, or use a separate scan for additional samples combined with the target sample, all of which can introduce delays and potentials for contamination or unintended variances between scans.

### Exemplary Materials

Exemplary systems and methods that are described herein may include various materials, such as vesicles and various components of the exemplary vesicles. Exemplary vesicles include one or more exemplary components, such as liposomes, solid lipid nanoparticles (LNPs), or nonlamellar lipid nanoparticles (e.g., inverted cubic and hexagonal phases), each of which are discussed in more detail below.

### A. Exemplary Components

Exemplary components may include solvents, lipids, surfactants, lipid nanoparticles (LNPs), micelles, stabilizers (e.g., hydrating agents such as glycerin or propylene glycol), buffers, salts, waxes and/or intermediate phases, each of which are discussed below.

Exemplary lipid nanoparticles (LNPs) or liposomes may include isopropyl myristate and/or cholesterol.

### 1. Exemplary Solvents

Exemplary solvents may include alcohols, ethers, sulfoxides, hydrocarbons, aromatic compounds, halogenated compounds (e.g., CHCl₃ and CCl₄). In some implementations, exemplary solvents may include ethanol.

Exemplary solvents do not include water, where water represents an aqueous contamination of a formulated vesicle.

### 2. Exemplary Lipids

Exemplary lipids may improve nanoparticle properties including particle stability, delivery efficacy, tolerability, biodistribution, or combinations thereof.

In some implementations, exemplary lipids may include triglycerides, fats, phospholipids, cationic lipids, PEGylated lipids, bio-ionizable lipids, phospholipids, cholesterols, waxes, steroids, or any combinations thereof.

In some implementations, exemplary lipids may include cholesterol, isopropyl myristate, and stearate.

In some implementations, exemplary lipids may include phosphotidylcholine and phosphati-dylethanolamine.

In some implementations, exemplary lipids may include (3060ᵢₗₒ), tetrakis (8-methylnonyl)-3,3′,3″,3′″-(((methylazanediyl)-bis(propane-3,1-diyl))bis(azanetriyl))tetrapropionate; (9A1P9), decyl (2-(dioctylammonio)ethyl) phosphate; (A2-Iso5-2DC18), ethyl 5,5-di((Z)-heptadec-8-en-1-yl)-1-(3-(pyrrolindin-1-yl)propyl)-2,5-dihydro-1H-imidazole-2-carboxylate; (ALC-0315), ((4-hydroxybutyl)azanediyl)bis(hexane-6,1-diyl)bis(2-hexyldecanoate); (ALC-0159), 2-[(polyethylene glycol)-2000]-N,N-ditetradecylacetamide; (β-sitosterol), (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; (BAME-O16B), bis(2-(dodecyldisulfanyl)ethyl) 3,3′-((3-methyl-9-oxo-10-oxa-13,14-dithia-3,6-diazahexacosyl)azanediyl)dipropionate; (BHEM-Cholesterol), 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1Hcyclopenta[a]phenanthren-3-yl)oxy)carbonyl)amino)-N,N-bis(2-hydroxyethyl)-Nmethylethan-1-aminium bromide; (C12-200), 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); (cKK-E12), 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; (DLin-MC3-DMA), (6Z,9Z,28Z,31Z)-heptatriaconta-6,9,28,31-tetraen-19-yl 4-(dimethylamino)butanoate; (DOPE), 1,2-diolcoyl-sn-glycero-3-phosphoethanolamine; (DOSPA), 2,3-dioleyloxy-N-[2-(sperminecarboxamido)ethyl]-N,N-dimethyl-1-propanaminiumtrifluoroacetate; (DOTAP), 1,2-dioleoyl-3-trimethylammonium-propane; (DOTMA), 1,2-di-O-octadecenyl-3-trimethylammonium-propane; (DSPC), 1,2-distearoyl-snglycero-3-phosphocholine; ePC, ethylphosphatidylcholine; (FTT5), hexa(octan-3-yl)9,9′,9″,9′″,9″″,9′″″-((((benzene-1,3,5-tricarbonyl)yris(azanediyl)) tris (propane-3,1-diyl))tris(azanetriyl))hexanonanoate; (Lipid H (SM-102)), heptadecan-9-yl 8-((2-hydroxyethyl)(6-oxo-6-(undecyloxy)hexyl)amino) octanoate; (OF-Deg-Lin), (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z, 12″Z,12′″Z)-tetrakis (octadeca-9,12-dienoate); (PEG2000-DMG), 1,2-dimyristoylrac-glycero-3-methoxypolyethylene glycol-2000; TT3, N1,N3,N5-tris(3-(didodecylamino)propyl)benzene-1,3,5-tricarboxamide; or combinations thereof.

In some aspects, the exemplary lipids may include (DSPC), 1,2 distearoyl-snglycero-3-phosphocholine and/or ePC, ethylphosphatidylcholine.

### 3. Exemplary Intermediate Phases

Exemplary intermediate phases of LNPs may include lamellar phases, hexagonal phases, bi-continuous cubic phases, a lipid nanoparticle (LNP), or combinations thereof.

### Exemplary Methods of Operation

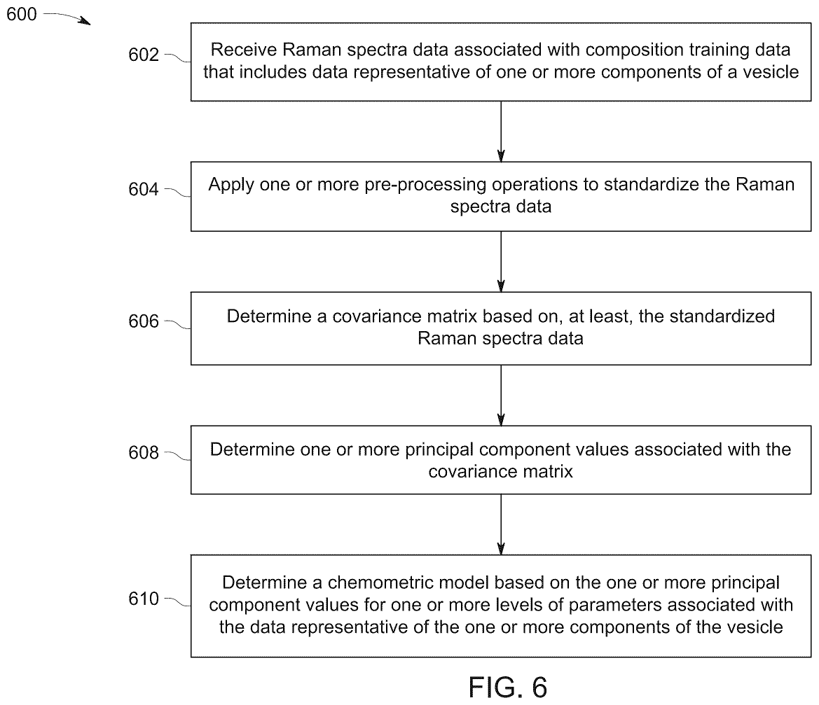

Referring now to FIG. 6, a flowchart illustrates a process 600 for determining a chemometric model, in accordance with some implementations of the present disclosure. Process 600 may be implemented using the spectroscopic system 110, as described above. The process 600 is described herein as being performed via the controller 111. However, it should be understood that the process 600 may be performed by one or more software and/or hardware components in various combinations and configurations. As illustrated in FIG. 6, the process 600 may include operations 602, 604, 606, 608, and 610. In some implementations, the process 600 is performed in the order as illustrated in FIG. 6.

In operation 602, the controller 111 receives Raman spectra data associated with composition training data that includes data representative of one or more components of a vesicle.

In operation 604, the controller 111 applies one or more pre-processing operations to standardize the Raman spectra data.

In some implementations, the controller 111 standardizes the Raman spectra data representative of the one or more components of the vesicle by applying one or more pre-processing operations to the Raman spectra data. In some implementations, the one or more pre-processing operations include region selection, spectra averaging, convolution filtering, 1^{st} derivative, 2^{nd} derivative, standard normal variate, multiplicative scatter correction, background removal, or any combinations thereof. The one or more standardized Raman spectrums associated with the composition training data are utilized to determine the chemometric model, as described below.

In some implementations, the controller 111 applies region selection pre-processing to the Raman spectra data representative of the one or more components of the vesicle. In some implementations, during region selection, part of the entire Raman spectra data is used. Using only a portion of the entire Raman spectra data may have advantages in certain aspects. For example, in some aspects, the very high and very low wavenumber regions of the Raman spectra data often feature a very low signal-to-noise ratio (SNR), which may present limited relevant information, and training on noisy data may result in overfitting of the data. In another example, in some aspects, distinguishing between different substances may be based on distinct regions of the Raman spectra data, where specific peaks (i.e., wavenumbers) may be observed. In such aspects, the remainder of the Raman spectra data may be observed as less relevant and the data representative of the remainder of the Raman spectra data may be discarded. In some implementations, region selection is a hyperparameter.

In some implementations, the controller 111 applies, at least, a second operation of pre-processing including Standard Normal Variate (SNV). During SNV scaling, each spectral datapoint is scaled with a standard normal transformation, as shown below in equation (2):

$${x}_{iSNV} = \frac{{x}_{i} - μ}{σ} \qquad ( 2 )$$

Where, xᵢ is the ith datapoint in the Raman spectra data, μ is the mean intensity of that spectrum, σ is the standard deviation of the intensity and x_{iSNV} is the corrected value for xᵢ.

In operation 606, the controller 111 determines a covariance matrix based on, at least, the standardized Raman spectra data.

The controller 111 determines a covariance matrix by utilizing the standardized Raman spectra to generate a P×P symmetric matrix. The covariance matrix may be a P-dimensional dataset, e.g., a 2-dimensional dataset, 2048×2048 symmetric matrix with 2048 variables.

In operation 608, the controller 111 determines one or more principal component values associated with the covariance matrix.

In some implementations, the controller 111 determines one or more eigenvectors and one or more eigenvalues based on the covariance matrix. The eigenvectors and eigenvalues are used to determine one or more principal component values. The controller 111 determines the one or more eigenvectors associated with the covariance matrix based on, at least, the direction of the axes where the variance of the Raman spectra data is greatest. The eigenvectors are commonly referred to as principal component values. The controller 111 determines one or more eigenvalues, where the eigenvalues are the coefficients associated with the eigenvectors, where the one or more eigenvalues may provide the amount of variance carried in each principal component value. The controller 111 organizes the eigenvectors from highest to lowest based on, at least, the one or more eigenvalues, which results in the principal component values being ordered based on their significance. In some implementations, the controller 111 determines the percent variance by dividing each eigenvalue by the sum of eigen values.

In operation 610, the controller 111 determines a chemometric model based on the one or more principal component values for one or more levels of parameters associated with the data representative of the one or more components of the vesicle.

In some implementations, the controller 111 determines a chemometric model including partial least squares regression (PLS) model, principal component analysis (PCA), principal component regression (PCR) model, least absolute shrinkage, selection operator (LASSO) model, clastic net regression model, support vector machine (SVM) model, or neural network model. In some implementations, the chemometric model includes a PLS model and/or a PCA model.

In some implementations, the controller 111 determines the chemometric model based on the one or more principal component values. The controller 111 determines a feature vector for the chemometric model by generating a matrix that has columns of the eigenvectors, as determined above. Based on, at least, the feature vector, the Raman spectra data representative of the one or more components of the vesicle is reoriented from the original axes to the axes represented by the one or more principal component values. As a result, the chemometric model is determined for one or more levels of parameters associated with the data representative of one or more components of the composition training data.

In some aspects of process 600, the one or more levels of parameters are representative of a quality of the one or more components of the vesicle, wherein one of the levels indicate an identity, as described above, of the one or more components of the vesicle modeled by the chemometric model.

In some aspects of process 600, the one or more levels of parameters are representative of a quantity of the one or more components of the vesicle, wherein one of the levels indicate an amount, as described above, of the one or more components of the vesicle modeled by the chemometric model.

In some aspects, operation 602 may also include, the controller 111 analyzes the composition training data and identifies (i) one or more components, (ii) one or more pre-processing operations, (iii) one or more parameters representative of a quality of the one or more components of a sample of a vesicle, and (iv) one or more parameters representative of a quality of the one or more components of a vesicle, associated with each respective composition training data.

In some aspects, operation 602 may also include, the composition training data having one or more levels of parameters representative of a quality of the one or more components of a vesicle. The composition training data includes one or more levels of parameters representative of a quantity of the one or more components of a vesicle. The plurality of composition training data includes any one of one or more components including solvents, lipids, surfactants, lipid nanoparticles (LNPs), micelles, intermediate phases of LNPs, or any combinations thereof. In some implementations, the lipid nanoparticle or liposome includes isopropyl myristate and cholesterol.

The composition training data includes any one of one or more experimental designs. The experimental designs include uniform design, factorial design, fractional factorial design, response surface design, random design, block design, Box-Wilson Central Composite Design, Box-Behnken Design, or combinations thereof. In some implementations, the plurality of composition training data includes uniform design. The one or more experimental designs are provided as a process for the composition training data for carrying out research in an objective and controlled environment. Where precision is maximized, and specific conclusions may be drawn regarding one or more hypothesis statements. The one or more experimental designs may establish the effect that one or more factors or independent variables have on one or more dependent variables. The composition training data is generated based on any one of the one or more experimental designs, where the composition training data is generated during a process in an objective and controlled environment.

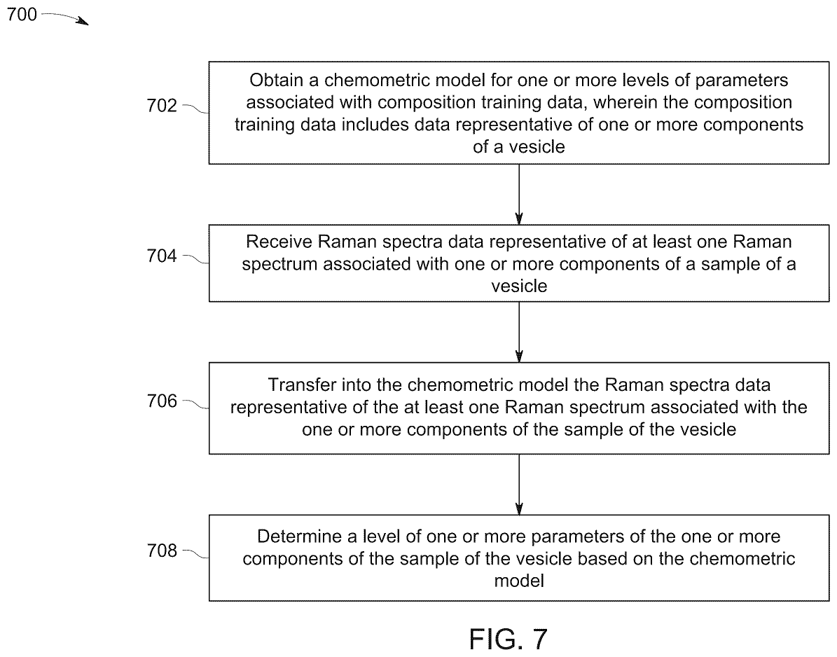

Referring now to FIG. 7, a flowchart illustrates a process 700 for determining a level of the one or more parameters of the sample of a vesicle, in accordance with some implementations of the present disclosure. Process 700 may be implemented using the spectroscopic system 110, as described above. The process 700 is described herein as being performed via the controller 111. However, it should be understood that the process 700 may be performed by one or more software and/or hardware components in various combinations and configurations. As illustrated in FIG. 7, the process 700 may include operation 702, 704, 706, and 708. In some implementations, the process 700 is performed in the order as illustrated in FIG. 7.

In operation 702, the controller 111 obtains a chemometric model for one or more levels of parameters associated with composition training data, wherein the composition training data includes data representative of one or more components of a vesicle.

In some implementations, the process 700 may also include, the chemometric model being obtained by the controller 111, where the controller 111 receives composition training data representative of the one or more components of the vesicle. The controller 111 determines the chemometric model for the one or more levels of parameters associated with the composition training data.

In operation 704, the controller 111 receives Raman spectra data representative of at least one Raman spectrum associated with one or more components of a sample of a vesicle.

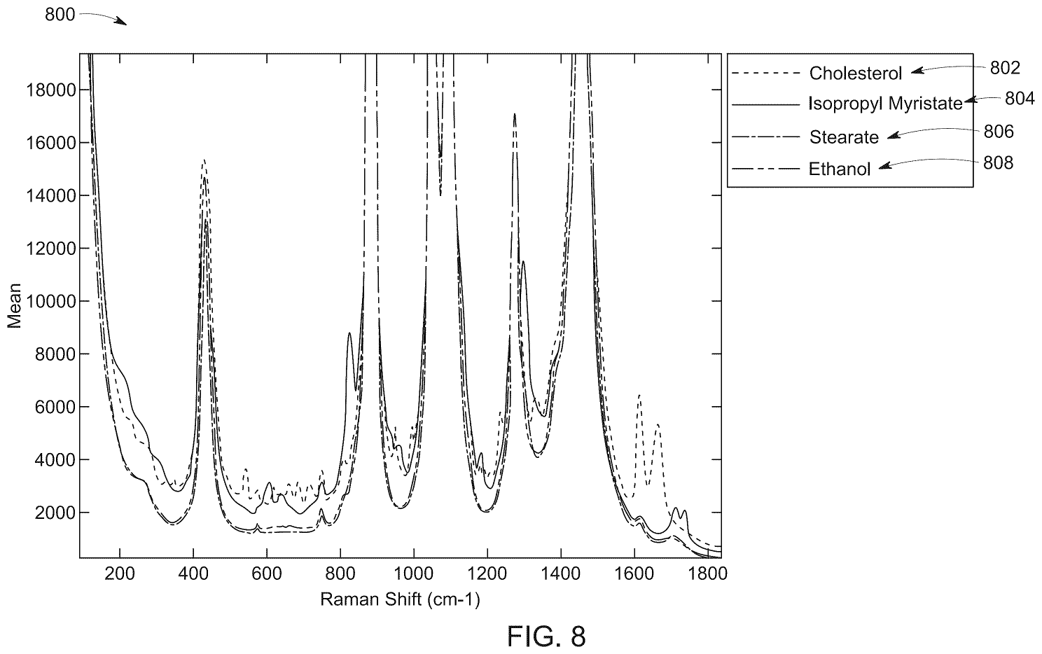

In some implementations, operation 704 may begin by performing scans of one or more samples of a vesicle, as discussed in detail above. The sample is scanned using, at least, the spectroscopic system 110, as described above in FIGS. 1-5. The spectroscopic system 110 directs a Raman laser beam (e.g., light), as described above, onto a surface or a focal point of a sample of a vesicle. For example, the Raman laser beam can be directed towards a container that includes a lipid vesicle, where the lipid vesicle comprises one or more components including a solvent, a lipid, a surfactant, a lipid nanoparticle (LNP), micelles, or an intermediate phase of a lipid nanoparticle (LNP). The resulting scattered light is directed back through the selective element 511 and the scattered light travels along the second beam path 520 and through the optical components 115 onto the detector 117. The resulting Raman spectra data of the sample is received by the detector 117, and signal processing and/or digitizing of the received Raman spectra data is handled by the electrical signal processors 113 (see, e.g., FIGS. 8, 10, 13 and 16-17 which illustrate exemplary Raman spectra data).

In some implementations, a scan of the sample of the vesicle is captured from 1 ms to 20 seconds exposure time. In some implementations, the scan of the sample of the vesicle captures both the bright and dark Raman spectra of the sample.

In operation 706, the controller 111 transfers into the chemometric model the Raman spectra data representative of the at least one Raman spectrum associated with the one or more components of the sample of the vesicle.

In some implementations, the controller 111 determines a covariance matrix based on, at least, the standardized Raman spectra data. The controller 111 determines a covariance matrix by utilizing the at least one standardized Raman spectra data to generate a P×P symmetric matrix. The covariance matrix may be a P-dimensional data set, e.g., a 2-dimensional dataset, 2048×2048 symmetric matrix with 2048 variables.

The controller 111 determines one or more eigenvectors and one or more eigenvalues based on the covariance matrix. The eigenvectors and eigenvalues are used to determine one or more principal component values. The controller 111 determines the one or more eigenvalues associated with the covariance matrix based on, at least, the direction of the axes where the variance of the Raman spectra data is greatest. The eigenvectors are commonly referred to as principal component values. The controller 111 determines one or more eigenvalues, where the eigenvalues are the coefficients associated with the eigenvectors, where the one or more eigenvalues may provide the amount of variance carried in each principal component. The controller 111 organizes the eigenvectors from highest to lowest based on, at least, the one or more eigenvalues, which results in the principal component values being ordered based on their significance. In some implementations, the controller 111 determines the percent variance by dividing each eigenvalue by the sum of eigenvalues.

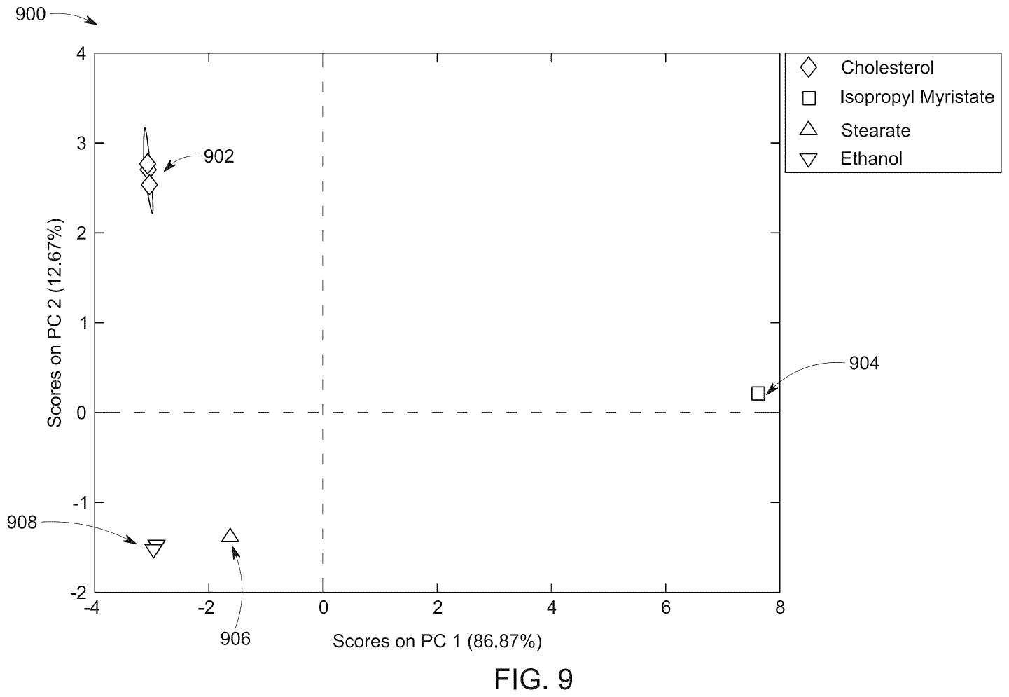

The controller 111 generates a graphical illustration representative of the chemometric model based on the one or more principal component values. The controller 111 determines a feature vector for the chemometric model by generating a matrix that has columns of the eigenvectors, as determined above. Based on, at least, the feature vector, the data associated with the plurality of Raman spectra is reoriented from the original axes to the axes represented by the one or more principal component values. As a result, a PCA model is determined for one or more levels of parameters associated with the data representative of one or more components of the composition training data (see, e.g., FIG. 9 that illustrates performance of an exemplary PCA model).

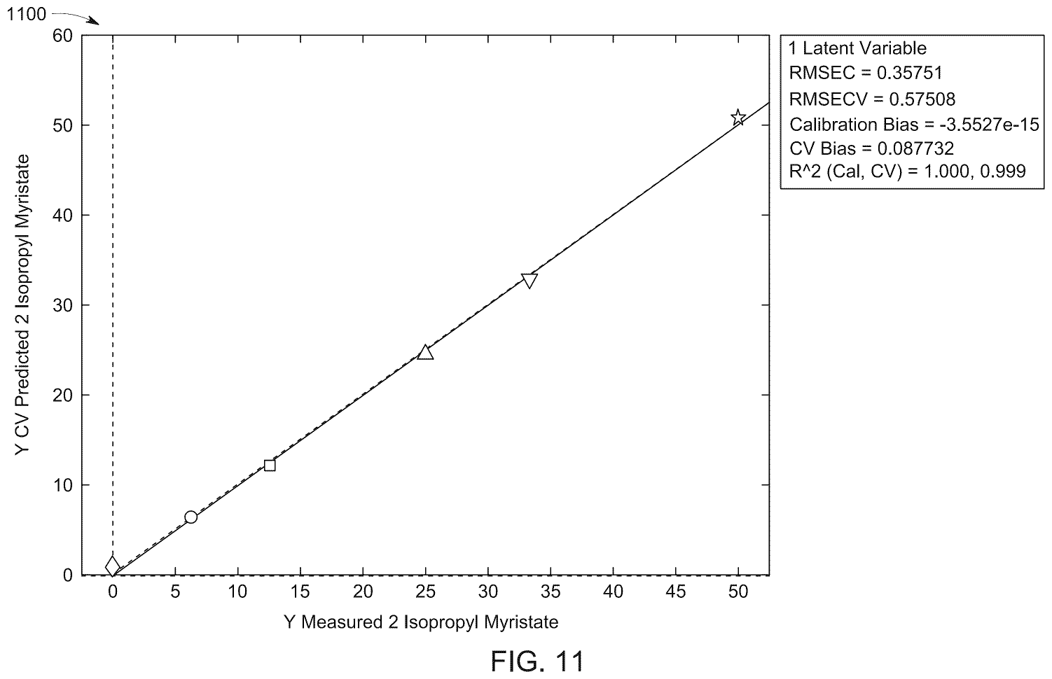

In some implementations, the controller 111 generates a graphical illustration representative of the chemometric model based on the one or more principal component values. The controller 111 determines a feature vector for the chemometric model by generating a matrix that has columns of the eigenvectors, as determined above. Based on, at least, the feature vector the data associated with the at least one Raman spectrum is reoriented from the original axes to the axes represented by the one or more principal component values. The controller 111 utilizes the principal component values to predict a vector and a full rank by one or more regressions. As a result, a PLS model is determined for one or more levels of parameters associated with the data representative of one or more components of the sample of the vesicle (sec, e.g., FIGS. 9 and 11 which illustrate exemplary PLS models).

In operation 708, the controller 111 determines a level of one or more parameters of the one or more components of the sample of the vesicle based on the chemometric model.

The controller 111 determines one or more parameters representative of a quality of the one or more components of the sample of the vesicle. The one or more parameters representative of the quality of the one or more components indicates an identity of the one or more components. In some implementations, the one or more components include one or more solvents, one or more lipids, one or more surfactants, one or more LNPs, one or more micelle, or one or more intermediate phases, as discussed in detail above.

In some implementations, the controller 111 determines one or more parameters representative of a quantity of the one or more components of the sample of the vesicle. The one or more parameters representative of the quality of the one or more components indicates an amount of the one or more components. In some implementations, the amount of the one or more components includes a concentration value or a ratio, as discussed above in detail.

In some aspects, process 700 may also include the one or more chemometric models includes any one of one or more of a PLS model, a PCA model, a PCR model, a least absolute shrinkage model, a LASSO model, an elastic net regression model, a SVM model, a neural network model, or any combinations thereof. In some implementations, the chemometric model includes a PCA model and any one of one or more of a PLS model, a PCR model, a least absolute shrinkage model, a LASSO model, an elastic net regression model, a SVM model, or any combinations thereof.

In some aspects, operation 704 may also include, the container being a reactor, a vessel, a sealed container configured for storage, a sealed container configured for transportation, or the like.

In some aspects, operation 704 may also include, the lipid vesicle being scanned directly with the spectroscopic system 110. The controller 111 analyzes the Raman spectra data of the sample of the vesicle and determines one or more components of the sample of the vesicle.

In some aspect, operation 704 may also include the controller 111 standardizing the Raman spectra data representative of the at least one Raman spectrum associated with the one or more components of the sample of the vesicle by applying one or more pre-processing operations to the Raman spectra data. In some implementations, the one or more pre-processing operations include region selection, spectra averaging, convolution filtering, 1^{st} derivative, 2^{nd} derivative, standard normal variate, multiplicative scatter correction, background removal, or any combinations thereof. The controller 111 applies region selection pre-processing to the Raman spectra data representative of the one or more components of the sample of the vesicle. In some implementations, during region selection, part of the entire Raman spectra data is used. Using only a portion of the entire Raman spectra data may have advantages in certain aspects. For example, the very high and very low wavenumber regions of the Raman spectra data often feature a very low signal-to-noise ratio (SNR), which may present limited relevant information, and training on noisy data may result in overfitting of the data. In another example, distinguishing between different substances may be based on distinct regions of the Raman spectra data, where specific peaks (i.e., wavenumbers) may be observed. In such aspects, the remainder of the Raman spectra data may be observed as less relevant and the data representative of the remainder of the Raman spectra data may be discarded. In some implementations, region selection is a hyperparameter. The controller 111 applies, at least, a second operation of pre-processing including Standard Normal Variate (SNV). During SNV scaling, each spectral datapoint is scaled with a standard normal transformation, as shown below in equation (2):

$${x}_{iSNV} = \frac{{x}_{i} - μ}{σ} \qquad ( 2 )$$

Where, xᵢ is the ith datapoint in the Raman spectra data, μ is the mean intensity of that spectrum, σ is the standard deviation of the intensity and x_{iSNV} is the corrected value for xᵢ.

In some aspects, operation 708 may also include, any one of the one or more parameters including concentration, fluorescence, refractive index, mass spectra, electrochemical behavior, or combinations thereof. As used herein the “level” of the parameter is a qualitive value or quantitative value corresponding to the parameter. For example, the parameter may be a concentration where the level is the value of the concentrations such as in grams per milliliter. As another example, the parameter may be a concentration where the level is selected from a qualitative value such as low, medium, and high.

The present disclosure illustrates a process of monitoring a production of lipid vesicles. In some implementations, the process of monitoring the production of lipid vesicles comprises combining at least one lipid with at least one solvent thereby forming a sample and monitoring the sample using the spectroscopic system 110, as described above with reference to FIGS. 6 and 7.

In some implementations, the process of monitoring the production of lipid vesicles comprises monitoring for a presence or an absence of one or more components of the lipid vesicle. The one or more components of the lipid vesicles includes a solvent, a lipid, a surfactant, a lipid nanoparticle (LNP), micelles, or an intermediate phase of a lipid nanoparticle (LNP).

In some implementations, the process of monitoring the production of lipid vesicles comprises monitoring for a quantity of one or more components of the lipid vesicle using the spectroscopic system 110, as described above with reference to FIGS. 6 and 7.

### Experimental Data

### A. Exemplary Chemometric Models

In some implementations, the experimental data described below in this section includes, at least, any one of the methods of operation described above in FIGS. 6-7. The performance of an exemplary PCA model is illustrated in FIG. 9 and the performance of an exemplary PLS model is illustrated by FIGS. 11, 14, and 15. The exemplary PCA and PLS models are the same models used with reference to FIGS. 6-7. The models are trained using composition training data including exemplary Raman spectra data (which may be actual data) and one or more exemplary known levels of a parameter of one or more components of a vesicle.

FIG. 8 is a graphical illustration 800 of Raman spectra data representative of at least one Raman spectrum associated with one or more components of a vesicle. As illustrated in the graphical illustration 800, Raman spectra data is provided for cholesterol 802, isopropyl myristate 804, stearate 806, and ethanol 808.

FIG. 9 is a graphical illustration 900 of the performance of the exemplary chemometric model using principal component analysis for determining one or more parameters representative of the quality of one or more components of a sample of a vesicle. In particular, FIG. 9 illustrates the identity of cholesterol 902, isopropyl myristate 904, stearate 906, and ethanol 908.

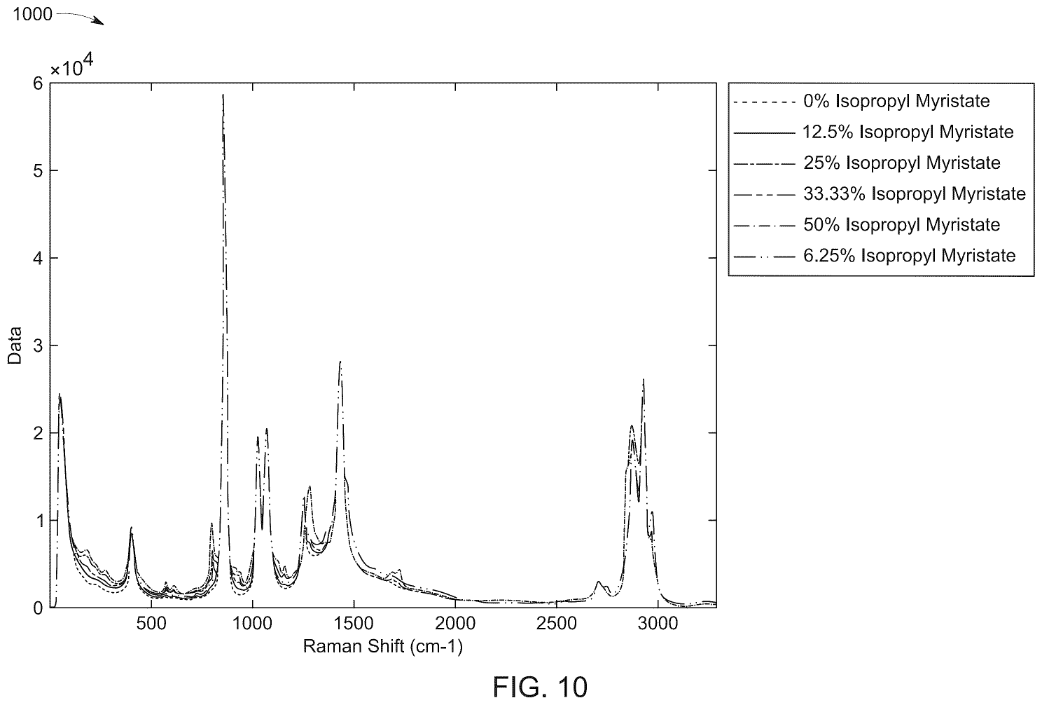

FIG. 10 is a graphical illustration 1000 of Raman spectra data of a mono-component lipid quantification in an ethanol solution.

FIG. 11 is a graphical illustration 1100 of the performance of the exemplary chemometric model using PLS for determining one or more parameters representative of a quantity of isopropyl myristate in ethanol. As illustrated in FIG. 11, the prediction correlation coefficient of cross validation (R²) has a value of 0.999 and an RMSEC has a value of 0.358 mg/mL. In addition, the RMSECV has a value of 0.575 mg/mL.

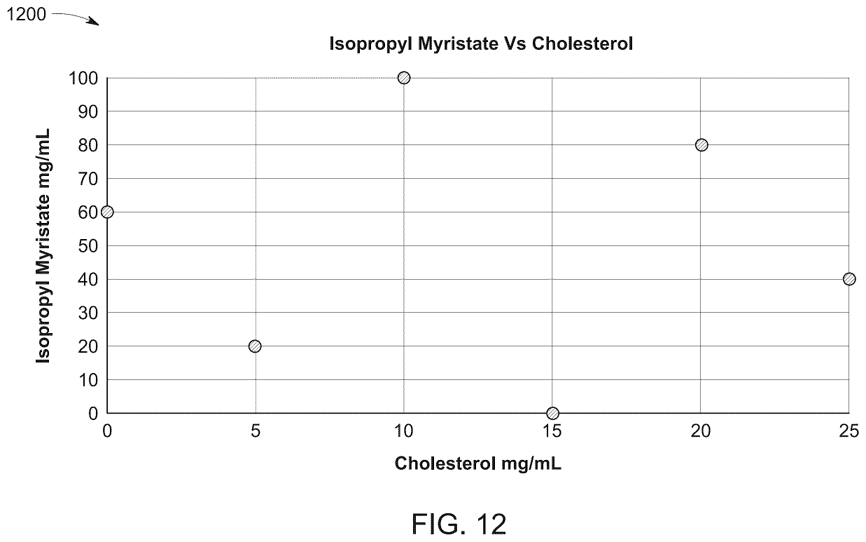

FIG. 12 shows a graphical illustration 1200 of Raman spectra data representative of the concentration of isopropyl myristate versus the concentration of cholesterol.

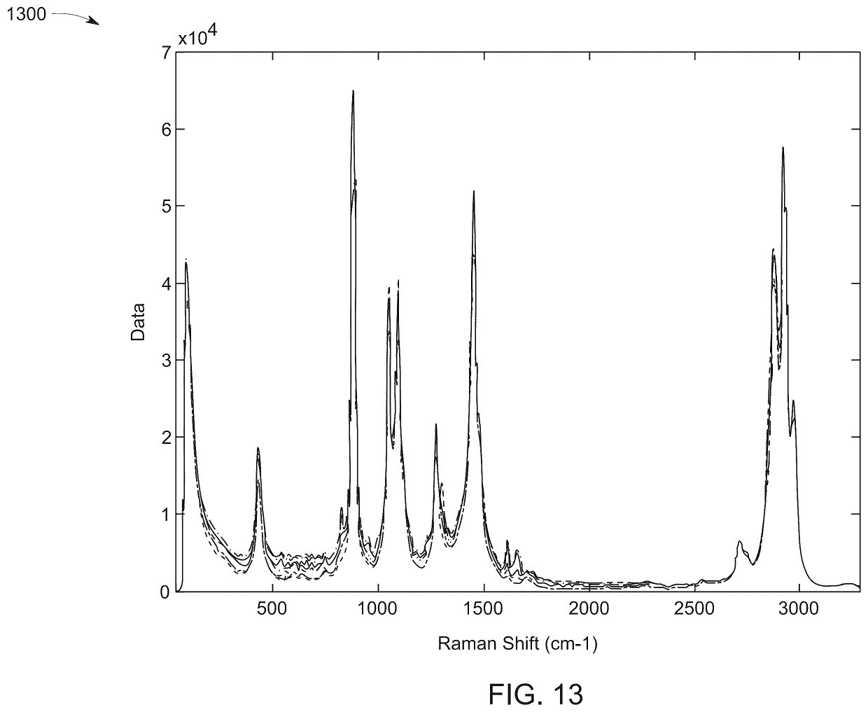

FIG. 13 shows a graphical illustration 1300 of Raman spectra data representative of at least one Raman spectrum associated with one or more components of a vesicle. The graphical illustration 1300 includes Raman spectra data for an aqueous contamination.

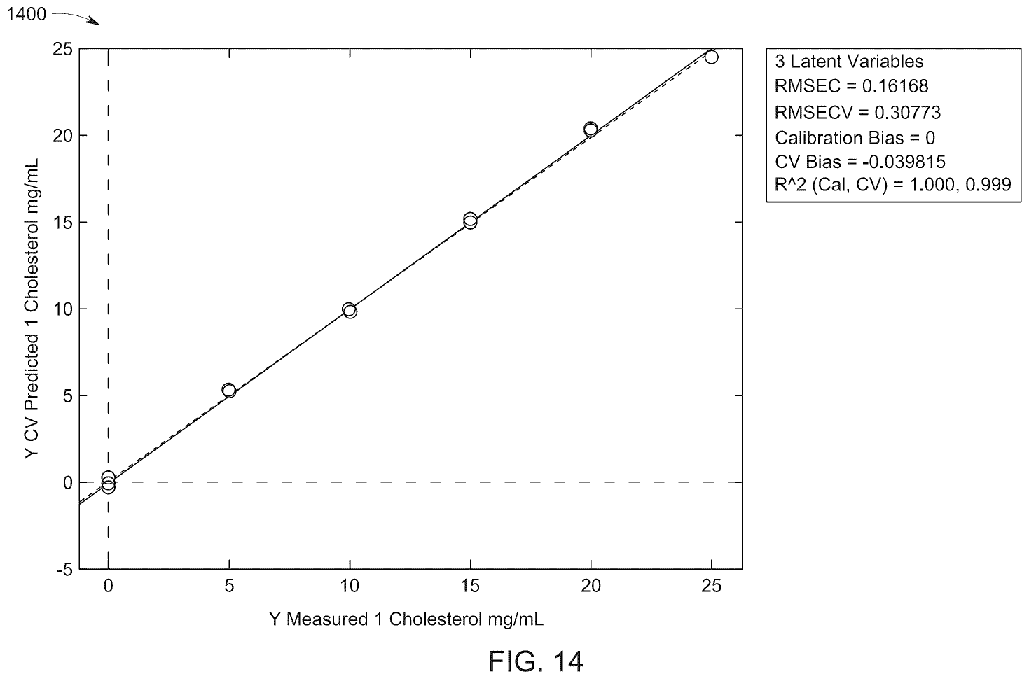

FIG. 14 is a graphical illustration 1400 of the performance of the exemplary chemometric model using PLS for determining one or more parameters representative of a quantity of isopropyl myristate and cholesterol in a mixture with ethanol. As illustrated in FIG. 14, a correlation coefficient (R²) has a value of 1.000, 1.000 and a RMSECV has a value of 0.308 mg/mL for cholesterol.

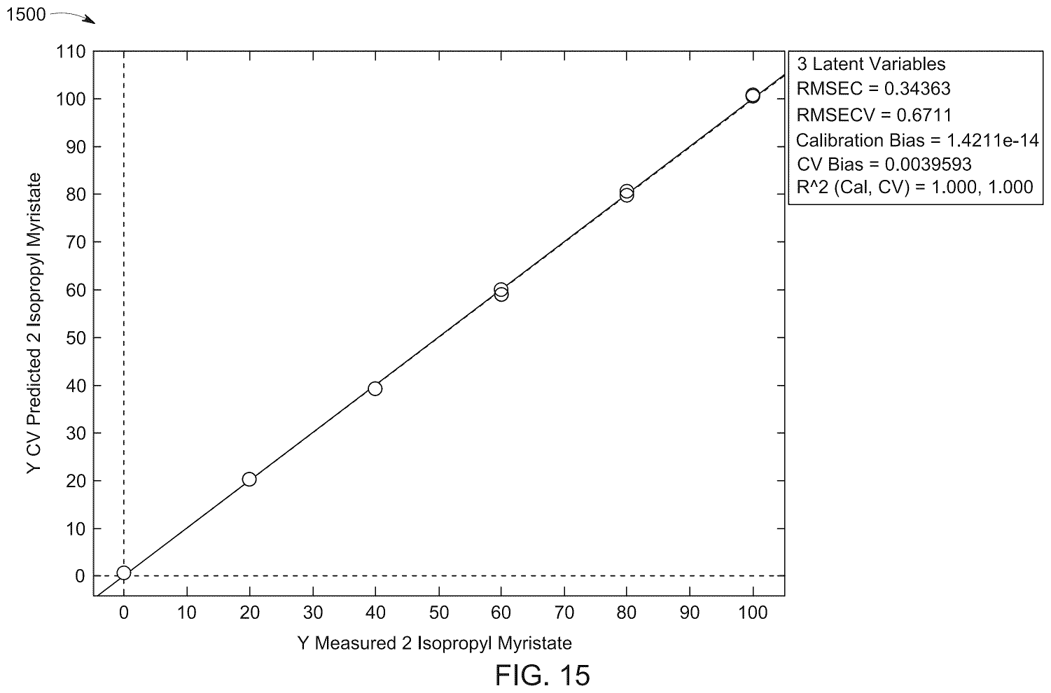

FIG. 15 is a graphical illustration 1500 of the performance of the exemplary chemometric model using PLS for determining one or more parameters representative of a quantity of isopropyl myristate and cholesterol in a mixture with ethanol. As illustrated in FIG. 15, a correlation coefficient (R²) has a value of 1.000, 1.000 and a RMSECV has a value of 0.671 mg/mL for isopropyl myristate.

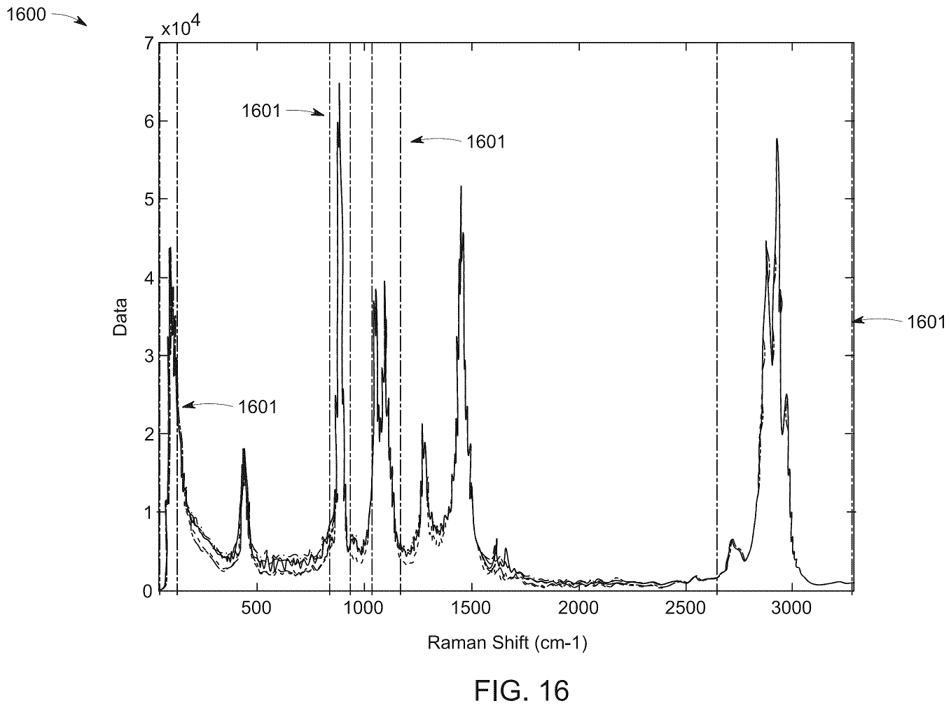

FIG. 16 is a graphical illustration 1600 of the Raman spectra data from the graphical illustration 1300, wherein the red bar regions 1601 represent data excluded from quantification models.

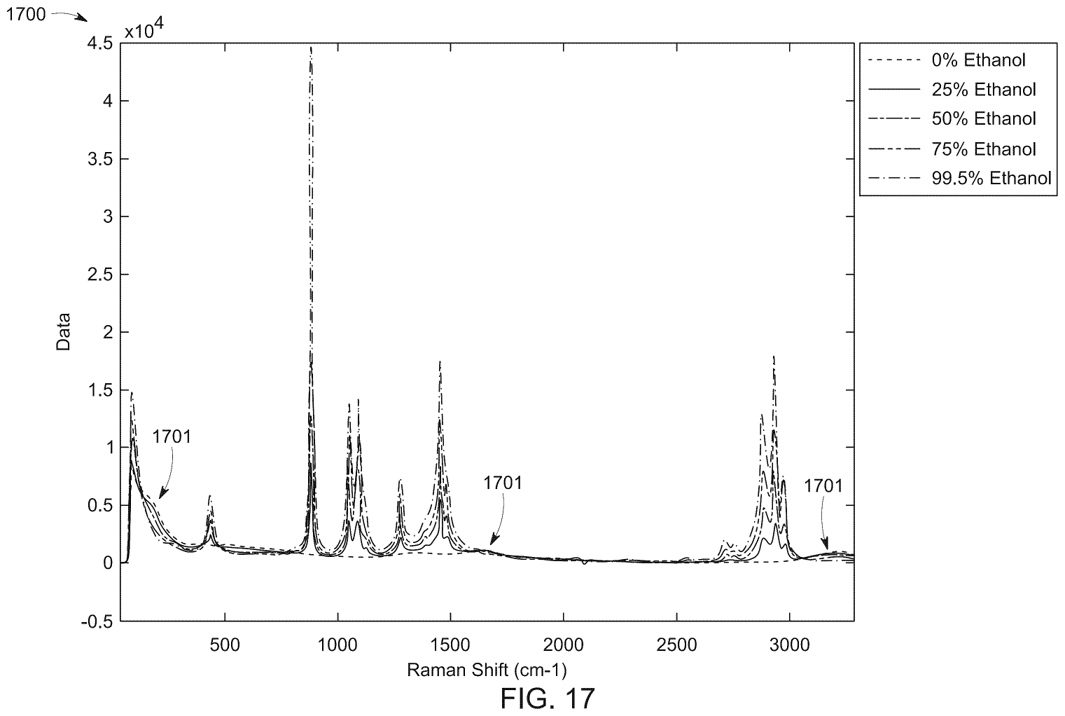

FIG. 17 is a graphical illustration 1700 of the Raman spectra data for aqueous contamination quantification (1701) acquired using the PLS model.

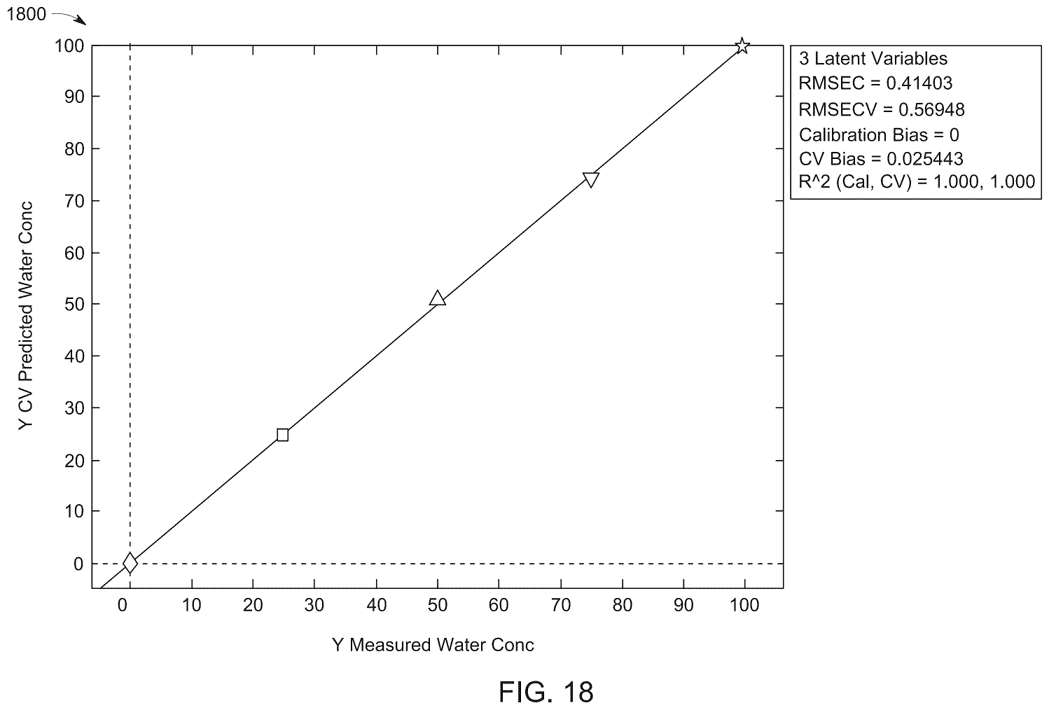

FIG. 18 is a graphical illustration 1800 of the performance of the exemplar chemometric model for determining one or more parameters representative of a quantity of water and, more specifically, a quantification of isopropyl myristate and cholesterol in a binary mixture in ethanol. The concentration of isopropyl myristate and cholesterol for creating training model was calculated using a uniform design. The PLS algorithm was used to develop the quantitative model. As illustrated in FIG. 18, a correlation coefficient (R²) of cross validation has a value of 1.000, and a REMSEC has a value of 0.56% v/v. The REMSECV value also has a value of 0.56948 mg/mL. Accordingly, based on the chemometric model the sample of the vesicle contained aqueous contamination can be quantified.

### B. Chemometric Modelling Data

For training the chemometric models described above, data representative of samples of vesicles containing one or more components were collected as described herein, where some additional details are described below.

For the identification of cholesterol, isopropyl myristate, and stearate, each component was dissolved in ethanol at 5 mg/mL concentration and the Raman spectra were collected. The PCA algorithm was applied on the collected Raman dataset to classify each component in the PCA space. The scores on each principal components would allow both identification as well as quantification.

For quantification of mono-component lipid, isopropyl myristate was mixed with ethanol at the concentration of 0, 6.5,12.5, 25,33.33, and 50% v/v. The Raman spectra was collected, and the dataset was fed into the PLS algorithm to create a PLS quantification models.

For a multi-component quantification in a mixture of isopropyl myristate and cholesterol, to cover the concentration levels of interest, a Uniform Design was used. The methods described herein do not require a designed experiment approach and can use randomly selected concentrations. The ranges for the designed experiments are indicated in Table 1 below.

TABLE 1

| | Isopropyl | Cholesterol | Isopropyl | Ethanol | Cholesterol | Isopropyl |
|---|---|---|---|---|---|---|
| Cholesterol | Meristate | mg/mL | Myristate | % | Volume | Myristate |
| 5 | 5 | 20 | 80 | 84 | 6.060606061 | 1.6 |
| 4 | 1 | 15 | 0 | 100 | 4.545454545 | 0 |
| 2 | 2 | 5 | 20 | 96 | 1.515151515 | 0.4 |
| 3 | 6 | 10 | 100 | 80 | 3.03030303 | 2 |
| 1 | 4 | 0 | 60 | 88 | 0 | 1.2 |
| 6 | 3 | 25 | 40 | 92 | 7.575757576 | 0.8 |

### C. Preprocessing and Chemometric Model Construction

Chemometric models, as discussed herein, were built using a partial least squares (PLS) model and/or a principal component analysis (PCA) model. The PLS model and the PCA model were modeled using MATLAB, as developed by MathWorks.

### Clauses

Implementations of the present disclosure are disclosed in the following clauses:
Clause 1. A computer-implemented method in an analytical instrument support apparatus, the method comprising:
   receiving, by one or more processors, Raman spectra data associated with composition training data, wherein the composition training data includes data representative of one or more components of a vesicle;
   applying, by one or more processors, one or more pre-processing operations to standardize the Raman spectra data;
   determining, by one or more processors, a covariance matrix based on, at least, the standardized Raman spectra data;
   determining, by one or more processors, one or more principal component values associated with the covariance matrix; and
   determining, by one or more processors, a chemometric model based on the one or more principal component values for one or more levels of parameters associated with the data representative of the one or more components of the vesicle.
Clause 2. The computer-implemented method according to Clause 1, wherein
   the one or more levels of parameters are representative of a quality of the one or more components of the vesicle, wherein one of the one or more levels indicate an identity of the one or more components of the vesicle modeled by the chemometric model; or
   the one or more levels of parameters are representative of a quantity of the one or more components of the vesicle, wherein one of the one or more levels indicate an amount of the one or more components of the vesicle modeled by the chemometric model.
Clause 3. The computer-implemented method according to either of Clause 1 or Clause 2, wherein the one or more components of the vesicle include a solvent, a lipid, a surfactant, a lipid nanoparticle (LNP), a micelles, or an intermediate phase of an LNP.
Clause 4. The computer-implemented method according to any one of Clauses 1-3, wherein one of the one or more components of the vesicle is a solvent including ethanol.
Clause 5. The computer-implemented method according to any one of Clauses 1-4, wherein one of the one or more components of the vesicle is a lipid including one or more cholesterol, isopropyl myristate, or stearate.
Clause 6. The computer-implemented method according to any one of Clauses 1-5, wherein one of the one or more components of the vesicle is a LNP including isopropyl myristate and cholesterol.
Clause 7. The computer-implemented method according to any one of Clauses 1-6, wherein the one or more components of the vesicle includes a solvent and a lipid, the composition training data including an experimental design for the formation of a LNP in an objective and controlled environment of concentrations for the lipid in the solvent.
Clause 8. The computer-implemented method according to any one of Clauses 1-7, wherein the experimental design is a uniform design.
Clause 9. The computer-implemented method according to any one of Clauses 1-8, wherein the chemometric model is a partial least squares regression (PLS) model and/or principal component analysis (PCA) model.
Clause 10. A computer-implemented method on an analytical instrument support apparatus, the method comprising:
   obtaining, by one or more processors, a chemometric model for one or more levels of parameters associated with composition training data, wherein the composition training data includes data representative of one or more components of a vesicle;
   receiving, by one or more processors, Raman spectra data representative of at least one Raman spectrum associated with one or more components of a sample of a vesicle;
   transferring into the chemometric model, by one or more processors, the Raman spectra data representative of the at least one Raman spectrum associated with the one or more components of the sample of the vesicle; and
   determining, by one or more processors, a level of one or more parameters of the one or more components of the sample of the vesicle based on the chemometric model.
Clause 11. The computer-implemented method according to Clause 10, the method further comprising:
scanning the sample of the vesicle with light directed from a Raman spectrometer;
in response to scanning the sample of the vesicle with the light, receiving, via a detector, scattered light directed back from the sample of the vesicle; and
in response to receiving, via the detector, the scattered light, generating a set of program instructions to communicate, by one or more processors, the scattered light to electrical signal processors,
wherein the electrical signal processors generate the Raman spectra data representative of the at least one Raman spectrum.
Clause 12. The computer-implemented method according to either of Clauses 10 or 11, wherein the chemometric model is provided by a method including:
   receiving, by one or more processors, the composition training data representative of the one or more components of the vesicle; and
   determining, by one or more processors, the chemometric model for the one or more levels of parameters associated with the composition training data.
Clause 13. The computer-implemented method according to any one of Clauses 10-12, wherein
   the level of the one or more parameters is representative of a quality of the one or more components, wherein the level of one of the one or more parameters indicates an identity of the one or more components determined by the chemometric model; or
   the level of the one or more parameters is representative of a quantity of the one or more components, wherein the level of one of the one or more parameters indicates an amount of the one or more components determined by the chemometric model.
Clause 14. The computer-implemented method according to any one of Clauses 10-13, wherein the one or more components includes a solvent, a lipid, a surfactant, a lipid nanoparticle (LNP), micelles, or an intermediate phase of an LNP.
Clause 15. One or more non-transitory computer-readable media having instructions stored thereon that, when executed by one or more processing devices of an analytical instrument support apparatus, cause the analytical instrument support apparatus to perform the computer-implemented method of Clause 1 or Clause 10.
Clause 16. An analytical instrument support system comprising:
   one or more processors;
   one or more non-transitory computer-readable storage media; and
   program instructions stored on at least one of the one or more non-transitory computer-readable storage media for execution by at least one of the one or more processors, the program instructions comprising steps for implementing the method of claim 1 or claim 10.
Clause 17. The analytical instrument support system according to Clause 16, wherein the program instructions are executed on a common computing device including at least one of the one or more processors.
Clause 18. The analytical instrument support apparatus of either of Clauses 16 or 17, wherein the program instructions are executed on a computing device including at least one of the one or more processors, and wherein the computing device is remote from an analytical instrument associated with the analytical instrument support system.
Clause 19. The analytical instrument support system of any one of Clauses 16-18. Wherein the program instructions are executed on a user computing device including at least one of the one or more processors.
Clause 20. The analytical instrument support system of any one of Clauses 16-19, wherein at least one of the one or more processors is disposed in an analytical instrument associated with the analytical instrument support system, and wherein the program instructions are executed on the at least one of the one or more processors.
Clause 21. An analytical instrument comprising:
   a light source configured to direct light onto a surface of a sample;
   a spectrograph configured to acquire a Raman spectrum from the surface of the sample in response to the light source directing light onto the surface of the sample;
   one or more processors;
   one or more non-transitory computer-readable storage media; and
   program instructions stored on at least one of the one or more non-transitory computer-readable storage media for execution by at least one of the one or more processors, wherein execution of the program instructions by at least one of the one or more processors cause the analytical instrument to implement the method of claim 1 or claim 10.
Clause 22. A method of monitoring a production of lipid vesicles, the method comprising combining at least one lipid with a solvent thereby forming a sample and monitoring the sample using the analytical instrument according to Clause 21.
Clause 23. The method according to Clause 22, wherein the sample is monitored for a presence or an absence of one or more components of the lipid vesicle.
Clause 24. The method according to Clause 22, wherein the sample is monitored for a quantity of one or more components of the lipid vesicle.

What is claimed is:

1. A computer-implemented method on an analytical instrument support apparatus, the method comprising:
receiving, by one or more processors, Raman spectra data associated with composition training data, wherein the composition training data includes data representative of one or more components of a vesicle;
applying, by one or more processors, one or more pre-processing operations to standardize the Raman spectra data;
determining, by one or more processors, a covariance matrix based on, at least, the standardized Raman spectra data;
determining, by one or more processors, one or more principal component values associated with the covariance matrix; and
determining, by one or more processors, a chemometric model based on the one or more principal component values for one or more levels of parameters associated with the data representative of the one or more components of the vesicle.

2. The computer-implemented method of claim 1, wherein
the one or more levels of parameters are representative of at least one selected from a group consisting of:
a quality of the one or more components of the vesicle, wherein one of the one or more levels indicate an identity of the one or more components of the vesicle modeled by the chemometric model, and
a quantity of the one or more components of the vesicle, wherein one of the one or more levels indicate an amount of the one or more components of the vesicle modeled by the chemometric model.

3. The method of claim 1, wherein the one or more components of the vesicle include at least one selected from a group consisting of a solvent, a lipid, a surfactant, a lipid nanoparticle (LNP), micelles, and an intermediate phase of an LNP.

4. The method of claim 3, wherein one of the one or more components of the vesicle is a solvent including ethanol.

5. The method of claim 3, wherein one of the one or more components of the vesicle is a lipid including one or more of cholesterol, isopropyl myristate, or stearate.

6. The method of claim 3, wherein one of the one or more components of the vesicle is a LNP including isopropyl myristate and cholesterol.

7. The method of claim 3, wherein the one or more components of the vesicle includes a solvent and a lipid, and the composition training data including an experimental design for formation of a LNP in an objective and controlled environment of concentrations for the lipid in the solvent.

8. The method of claim 7, wherein the experimental design is a uniform design.

9. The method of claim 1, wherein the chemometric model is at least one selected from a group consisting of a partial least squares regression (PLS) model and a principal component analysis (PCA).

10. A computer-implemented method on an analytical instrument support apparatus, the method comprising:
obtaining, by one or more processors, a chemometric model for one or more levels of parameters associated with composition training data, wherein the composition training data includes data representative of one or more components of a vesicle;
receiving, by one or more processors, Raman spectra data representative of at least one Raman spectrum associated with one or more components of a sample of a vesicle;
transferring into the chemometric model, by one or more processors, the Raman spectra data representative of the at least one Raman spectrum associated with the one or more components of the sample of the vesicle; and
determining, by one or more processors, a level of one or more parameters of the one or more components of the sample of the vesicle based on the chemometric model.

11. The method according to claim 10, the method further comprising:
scanning the sample of the vesicle with light directed from a Raman spectrometer;
in response to scanning the sample of the vesicle with the light, receiving, via a detector, scattered light directed back from the sample of the vesicle; and
in response to receiving, via the detector, the scattered light, generating a set of program instructions to communicate, by one or more processors, the scattered light to an electrical signal processor,
wherein the electrical signal processor generates the Raman spectra data representative of the at least one Raman spectrum.

12. The method according to claim 10, wherein the chemometric model is obtained by a method including:
receiving, by one or more processors, the composition training data representative of the one or more components of the vesicle; and
determining, by one or more processors, the chemometric model for the one or more levels of parameters associated with the composition training data.

13. The method of claim 10, wherein
the level of the one or more parameters is representative of at least one selected from a group consisting of:
a quality of the one or more components, wherein the level of one of the one or more parameters indicates an identity of the one or more components determined by the chemometric model, and
a quantity of the one or more components, wherein the level of one of the one or more parameters indicates an amount of the one or more components determined by the chemometric model.

14. The method according to claim 13, wherein the one or more components includes at least one selected from a group consisting of a solvent, a lipid, a surfactant, a lipid nanoparticle (LNP), micelles, and an intermediate phase of an LNP.

15. An analytical instrument support system comprising:
a light source configured to direct light onto a surface of a sample;
a spectrograph configured to acquire a Raman spectrum from the surface of the sample in response to the light source directing light onto the surface of the sample;
one or more processors;
one or more non-transitory computer-readable storage media; and
program instructions stored on at least one of the one or more non-transitory computer-readable storage media for execution by at least one of the one or more processors, wherein execution of the program instructions by at least one of the one or more processors cause the analytical instrument support system to:
obtain a chemometric model for one or more levels of parameters associated with composition training data, wherein the composition training data includes data representative of one or more components of a vesicle,
receive Raman spectra data representative of at least one Raman spectrum associated with one or more components of the sample,
transfer into the chemometric model the Raman spectra data representative of the at least one Raman spectrum associated with the one or more components of the sample of the vesicle, and
determine a level of one or more parameters of the one or more components of the sample based on the chemometric model.

16. The analytical instrument support system according to claim 15, wherein the one or more components includes at least one selected from a group consisting of a solvent, a lipid, a surfactant, a lipid nanoparticle (LNP), micelles, and an intermediate phase of an LNP.

17. The analytical instrument support system according to claim 15, wherein the program instructions are executed on a computing device including at least one of the one or more processors, and wherein the computing device is remote from an analytical instrument associated with the analytical instrument support system.

18. The analytical instrument support system according to claim 15, wherein the program instructions are executed on a user computing device including at least one of the one or more processors.

19. The analytical instrument support system according to claim 15, wherein at least one of the one or more processors is disposed in an analytical instrument associated with the analytical instrument support system, and wherein the program instructions are executed on the at least one of the one or more processors.

20. The analytical instrument support system according to claim 15, wherein
the level of the one or more parameters is representative of at least one selected from a group consisting of:
a quality of the one or more components, wherein the level of one of the one or more parameters indicates an identity of the one or more components determined by the chemometric model, and
a quantity of the one or more components, wherein the level of one of the one or more parameters indicates an amount of the one or more components determined by the chemometric model.
